(19) United States
(12) Patent Application Publication
(10) Pub. No.: US 2004/0234542 A1
(43) Pub. Date: Nov. 25, 2004

(54) Recombinant orf2 proteins of the swine hepatitis e virus and their use as a vaccine and as a diagnostic reagent for medical and veterinary applications

(76) Inventors: Xiang-Jin Meng, Blacksburg, VA (US); Robert H Purcell, Boyds, MD (US); Suzanne U Emerson, Kensington, MD (US)

Correspondence Address: NATIONAL INSTITUTES OF HEALTH, OFFICE OF TECHNOLOGY TRANSFER, 6011 EXECUTIVE BLVD SUITE 325, ROCKVILLE, MD, 20852-3804, US

(21) Appl. No.: 10/476,777

(22) PCT Filed: May 2, 2002
(86) PCT No.: PCT/US02/14100

Related U.S. Application Data
(60) Provisional application No. 60/289,220, filed on May 7, 2001.

(51) Int. Cl. A61K 39/29; C07K 14/02

(52) U.S. Cl. 424/189.1; 530/350

(57) ABSTRACT

The invention relates to open reading frame 2 (ORF-2) proteins of a swine hepatitis E virus and the use of these proteins as an antigen in diagnostic immunoassays and/or as immunogen or vaccine to protect against infection by hepatitis E.

### FIELD OF INVENTION

The invention is in the field of hepatitis virology. More specifically, this invention relates to recombinant ORF2 proteins derived from a swine hepatitis E virus and to diagnostic methods and vaccine applications which employ these proteins.

### BACKGROUND OF INVENTION

Hepatitis E virus (HEV), the causative agent of hepatitis E, is an important public health problem in developing countries. Most global public health organizations consider hepatitis E to be the major cause of acute viral hepatitis in young adults in regions where sanitation conditions are poor. The mortality rate of HEV infection is generally low, but was reportedly up to 20% in patients infected during pregnancy. In the United States, two cases of acute hepatitis E not associated with travel to present regions have been recently reported, and hepatitis E is now considered to be endemic in the United States. A vaccine for hepatitis E is not available yet. The first animal strain of HEV, swine hepatitis E virus (swine HEV), was recently identified and found to be ubiquitous in the general pig population in the United States and other countries, and to experimentally infect non-human primates, the surrogates of humans. The complete genome of swine HEV, including the putative capsid gene (ORF2), has been sequenced.

The possibility that swine HEV may infect humans raises a potential public health concern for zoonosis or xenozoonosis in the United States and perhaps other countries. Therefore, diagnostic reagents based on recombinant proteins of swine HEV will be very useful in screening donor pigs used in xenotransplantation and in detecting swine HEV or similar virus infection in humans. The diagnostic reagents may also be useful for veterinary studies and monitoring pig herds in general. A vaccine based on the recombinant capsid protein of swine HEV might also be useful in protecting humans against zoonotic and other HEV infections and pigs against infection with the swine HEV.

### SUMMARY OF INVENTION

The invention relates to isolated and substantially purified open reading frame 2 proteins encoded by the swine HEV genome and in particular to a recombinantly produced ORF2 protein consisting of amino acids 112-602 of the swine ORF2.

It is therefore an object of this invention to provide synthetic nucleic acid sequences capable of directing production of these recombinant HEV proteins, as well as equivalent natural nucleic acid sequences. Such natural nucleic acid sequences may be isolated from a cDNA or genomic library from which the gene capable of directing synthesis of the HEV proteins may be identified and isolated. For purposes of this application, nucleic acid sequence refers to RNA, DNA, cDNA or any synthetic variant thereof.

The invention also relates to methods of preparing the HEV proteins by expressing the recombinant protein in a host cell.

The invention also relates to the use of the resultant recombinant HEV proteins as diagnostic agents and as vaccines.

The present invention also encompasses methods of detecting antibodies specific for swine hepatitis E virus in biological samples. Such methods are useful in diagnosis of infection and disease caused by swine HEV, and for monitoring the progression of such disease. Such methods are also useful for monitoring the efficacy of therapeutic agents during the course of treatment of HEV infection and disease in a mammal.

### DETAILED DESCRIPTION OF THE FIGURES

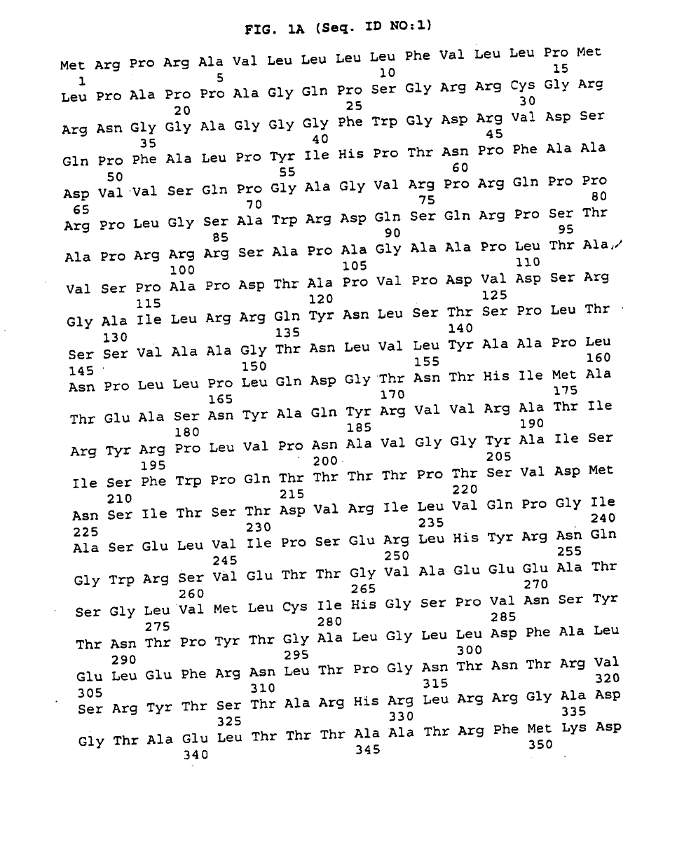

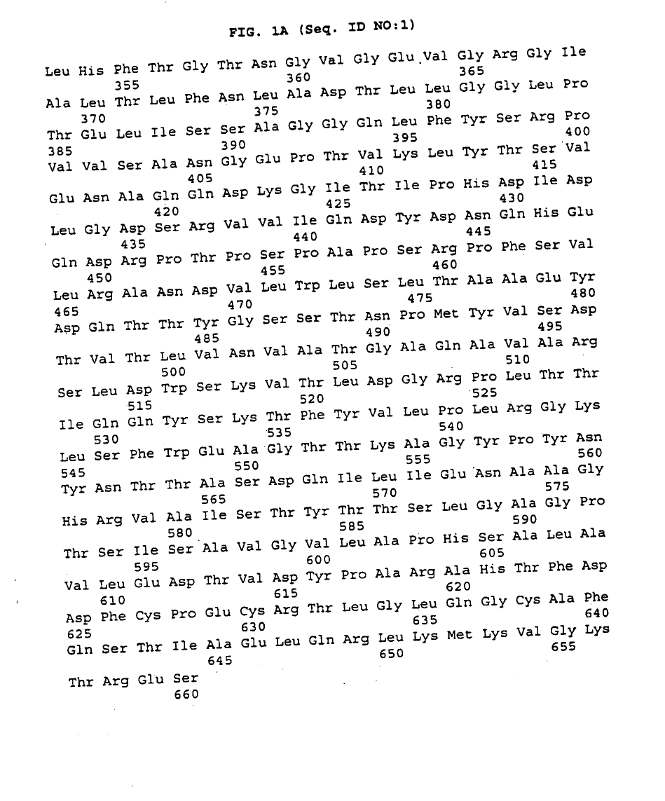

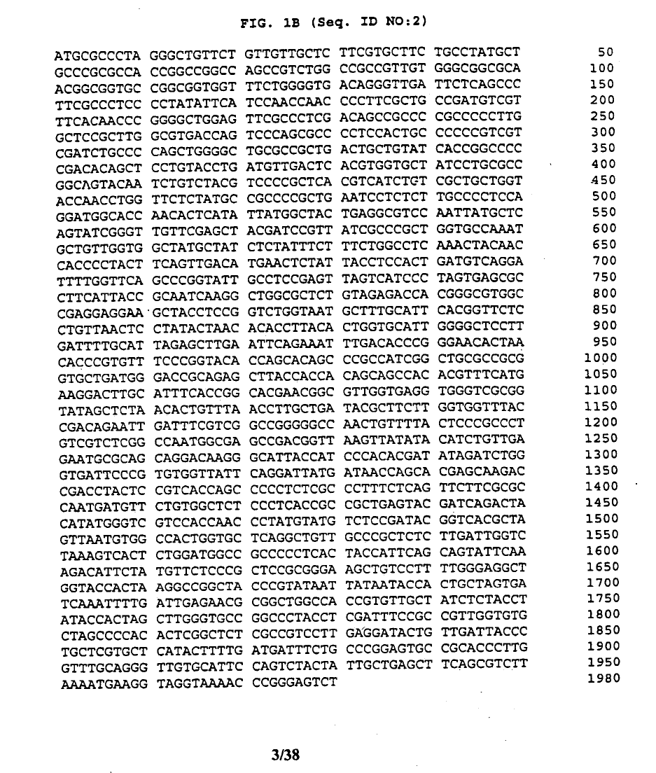

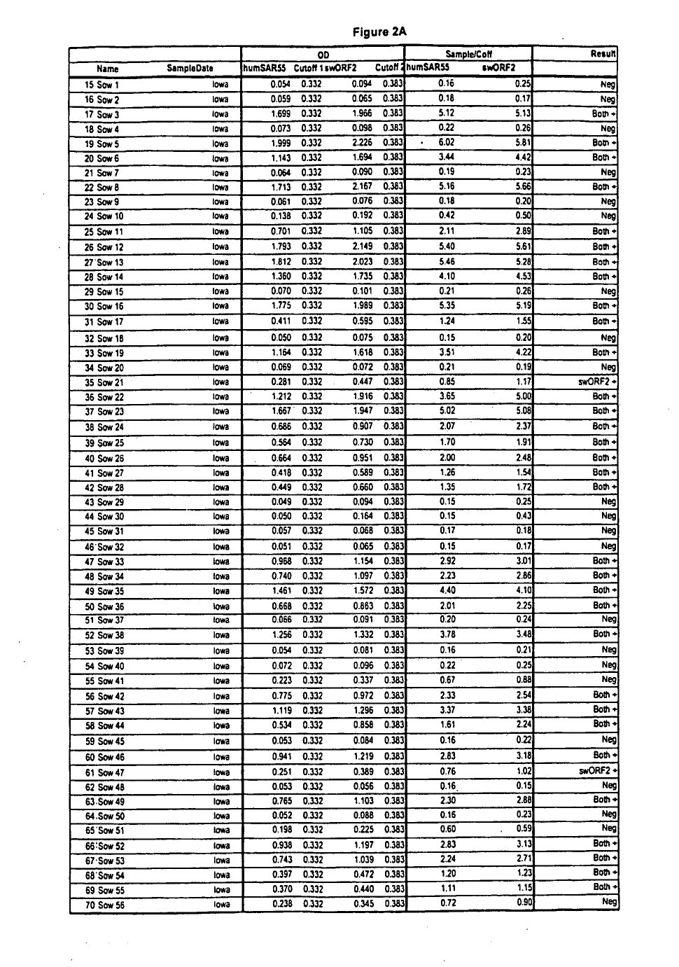

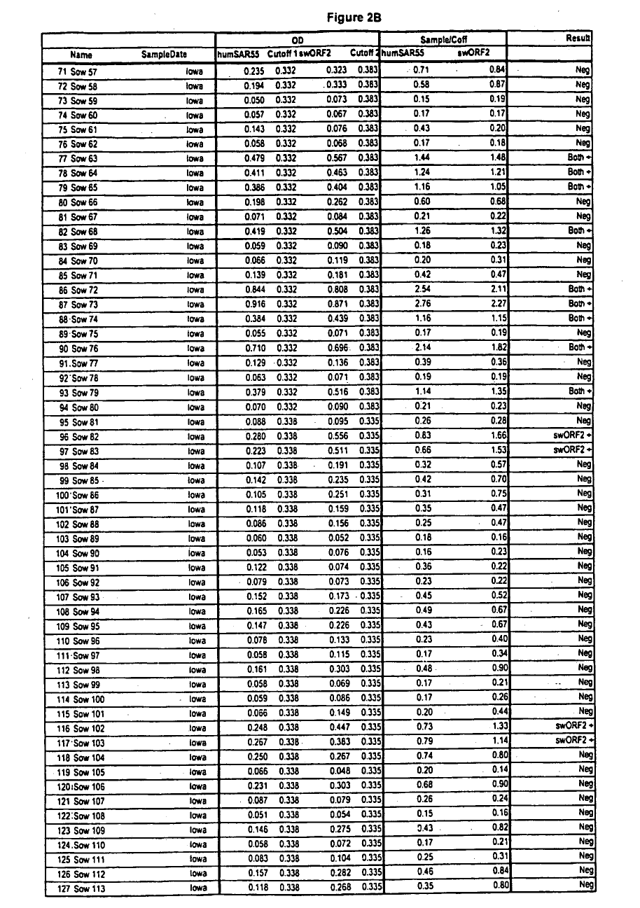

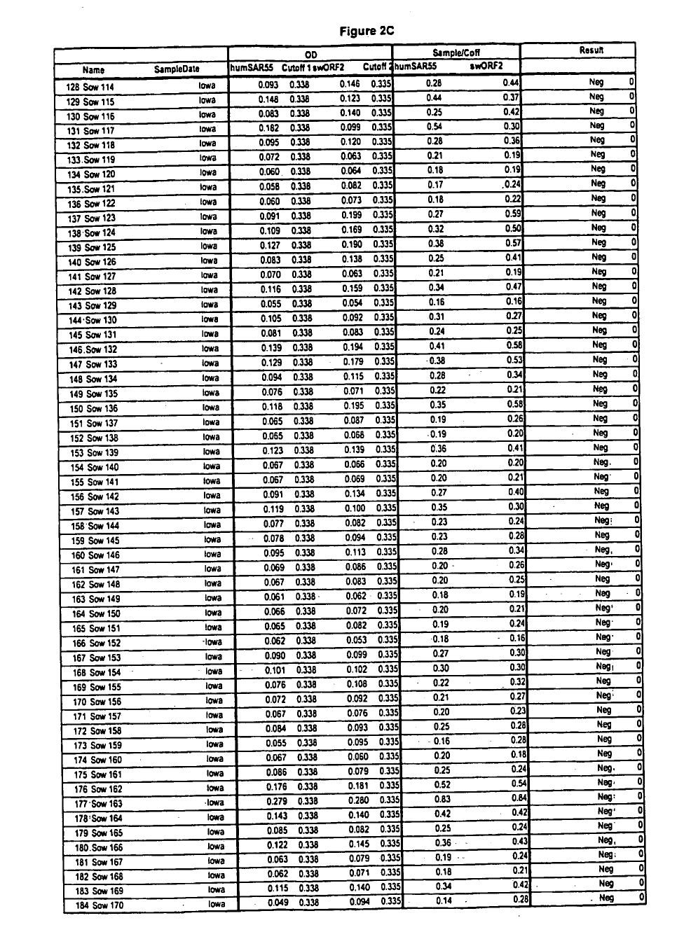

FIGS. 1A and 1B show amino acid (SEQ. ID NO:1, FIG. 1A) and nucleotide (SEQ. ID NO:2, FIG. 1B) sequences respectively of open reading frame 2 of the swine HEV of Meng et al. [Proc Natl Acad. Sci. USA (1997) 98:9860-9865]

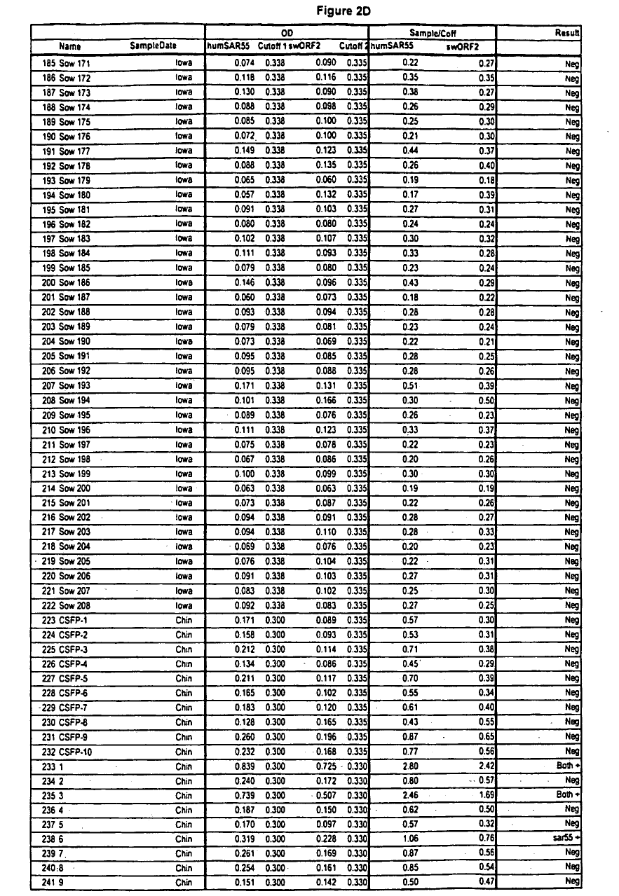

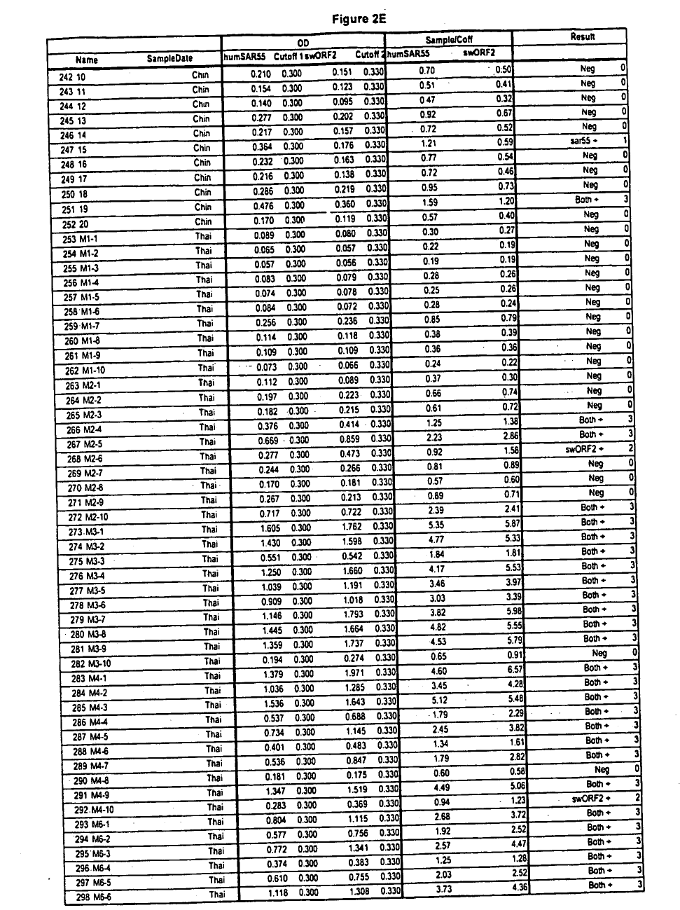

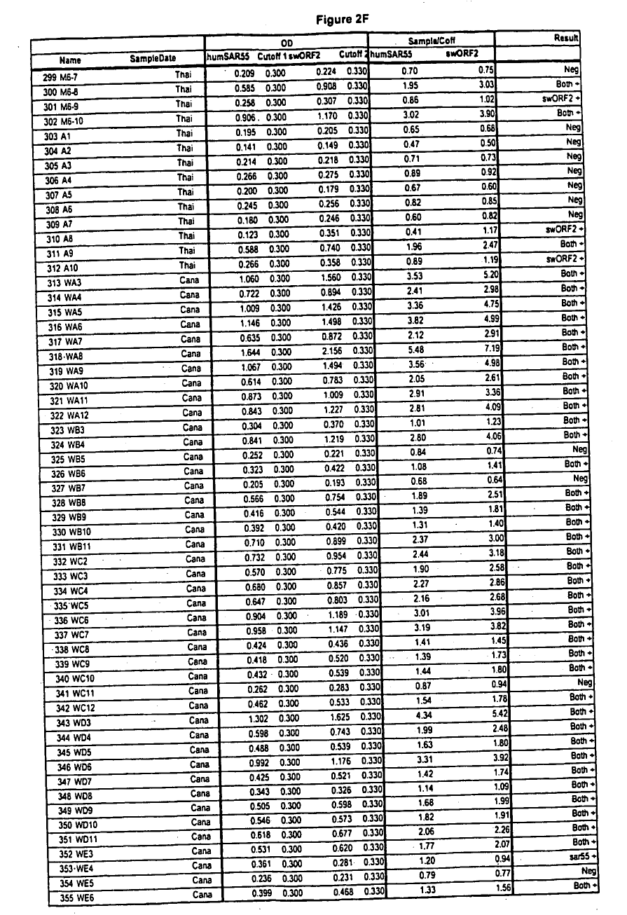

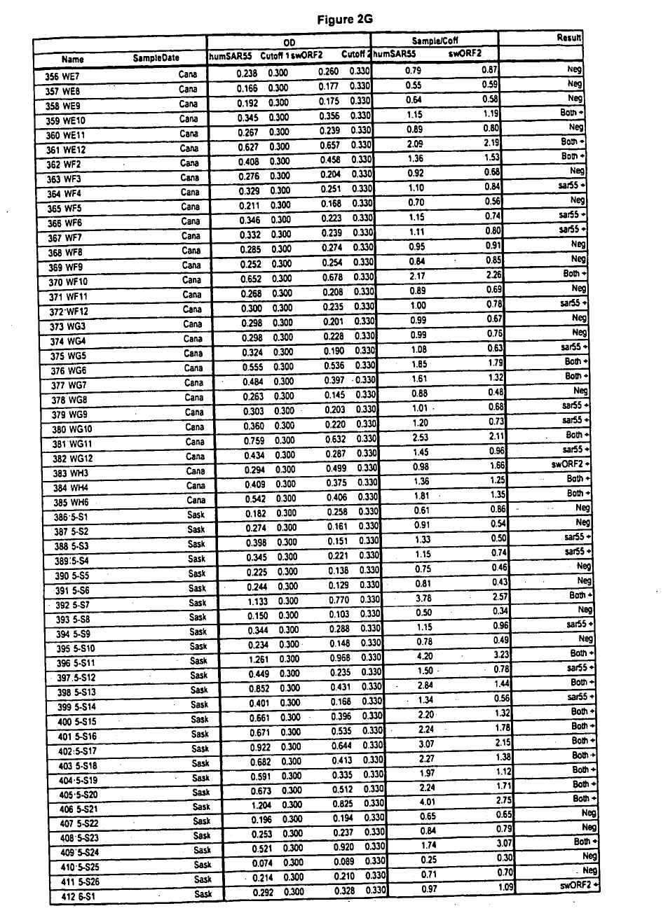

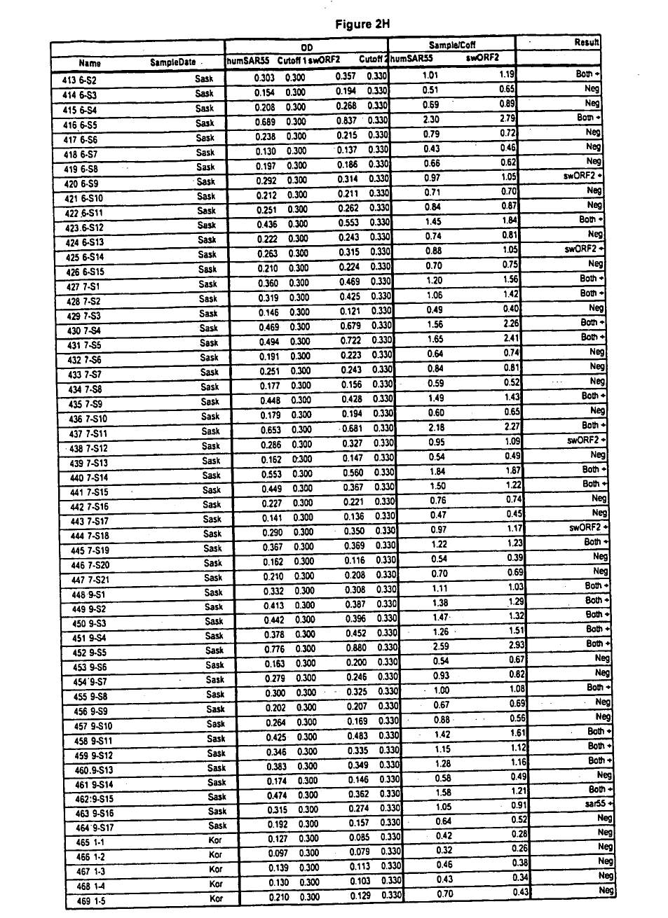

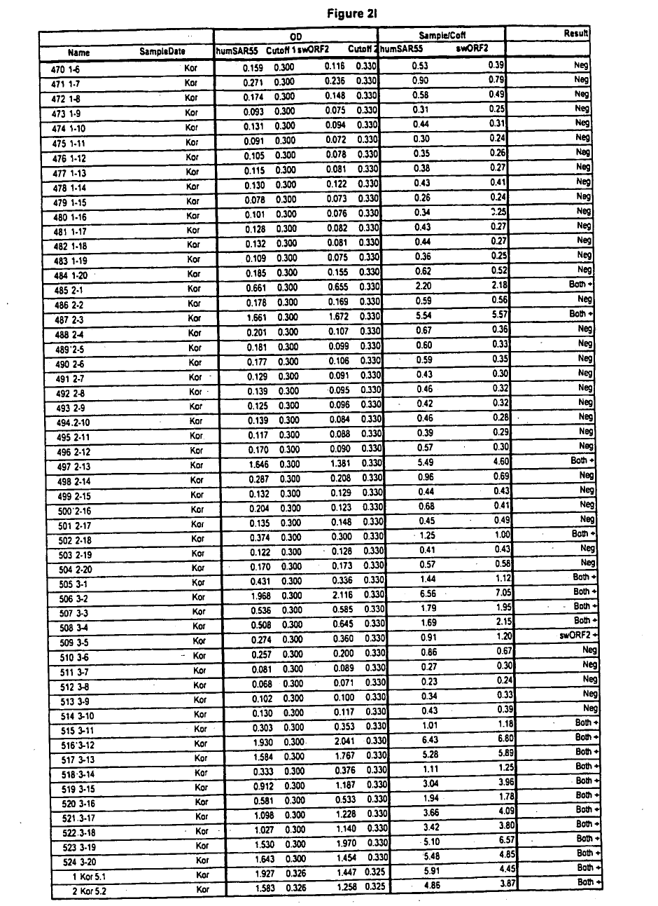

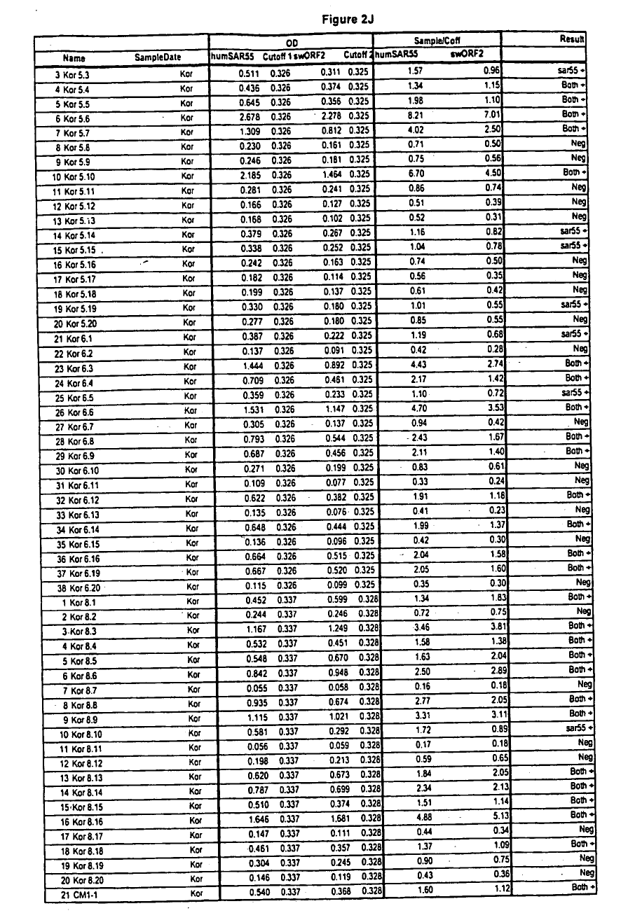

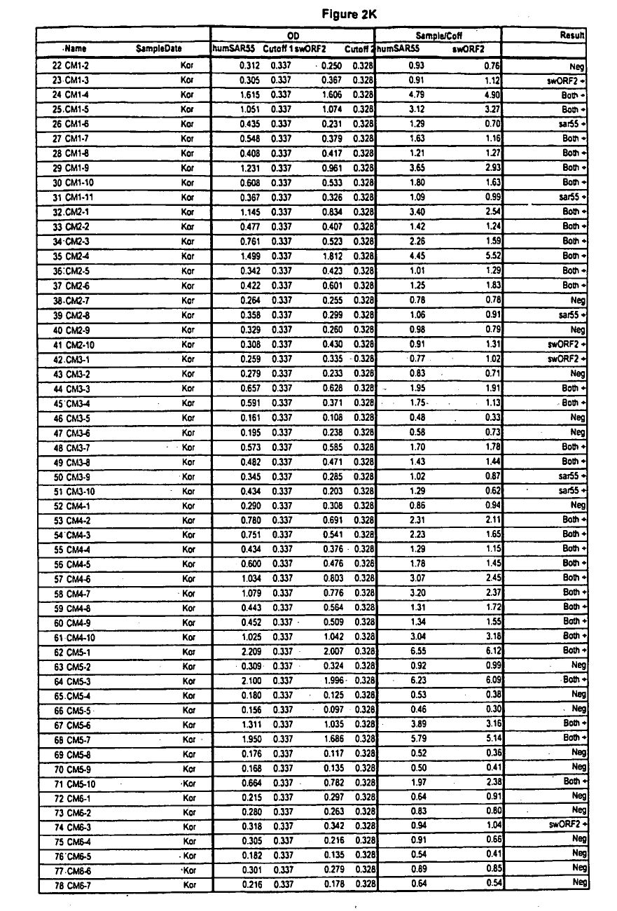

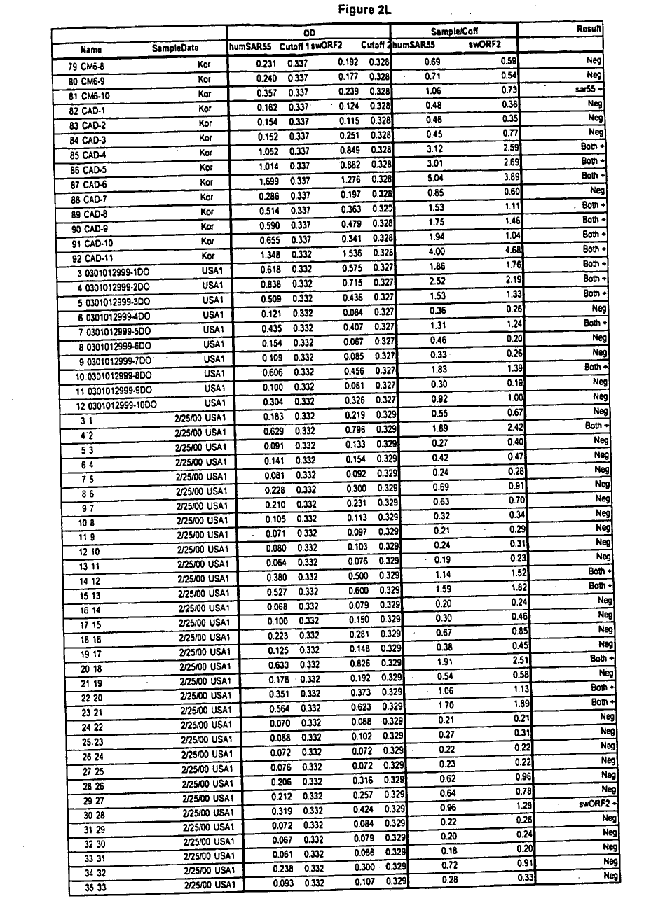

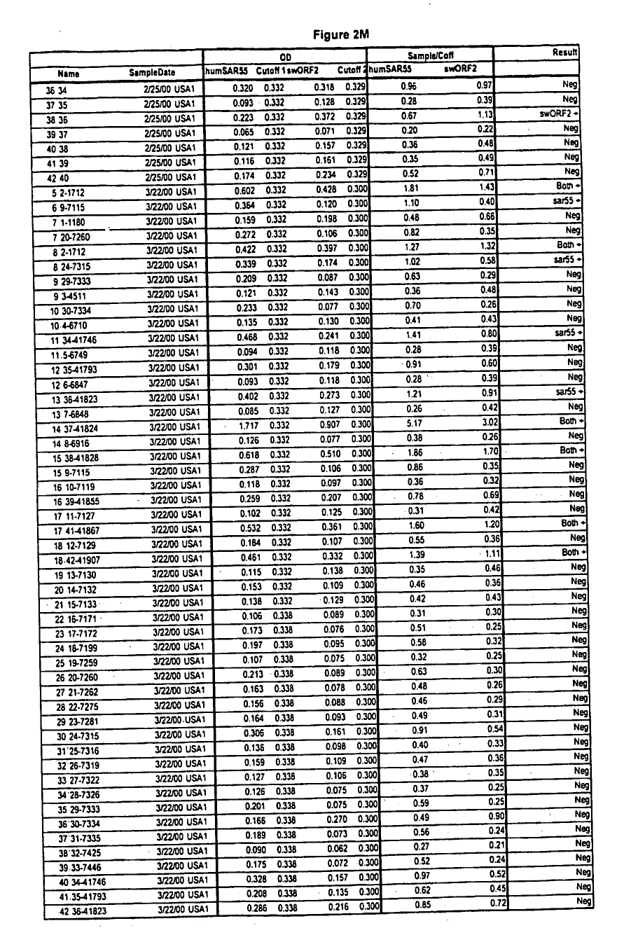

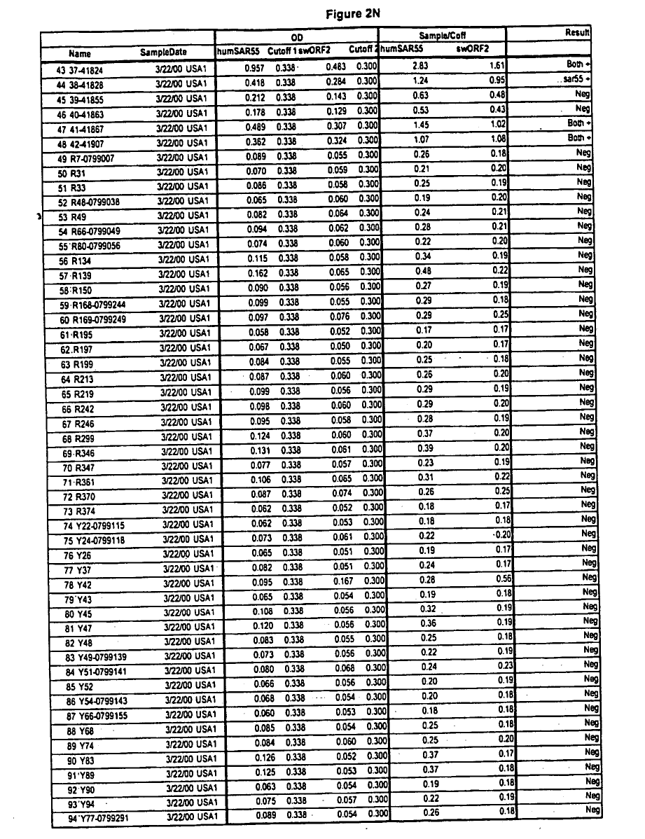

FIGS. 2A-2O show the results of EIAs, using as the antigen, either the swine ORF2 protein consisting of amino acids 112-602 of swine ORF2 (designated “swORF2” in the Figures) or the human HEV ORF2 antigen consisting of amino acids 112-607 of the ORF2 of the Pakistani SAR-55 strain of HEV (designated “humSAR55” in the Figures). Anti-HEV antibody levels were measured in serum from swine obtained from the United States (Iowa), China, Thailand, Canada and Korea (FIGS. 2A-2N) and the results of the EIAs with the swORF2 and humSAR55 antigens are summarized in FIG. 2O. In FIGS. 2A-2N a sample was considered positive if the ratio (see column headed “sample/coff”) of the optical density measured for the human SAR55 (“humSAR55” column) or swine antigens (“swORF2” column) to the cutoff value (see columns headed“cutoff”) for the humSAR55 or swORF2 antigens was greater than 1.0.

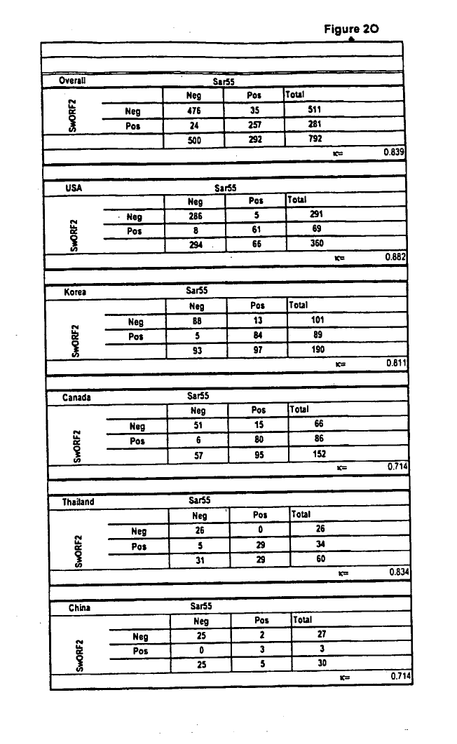

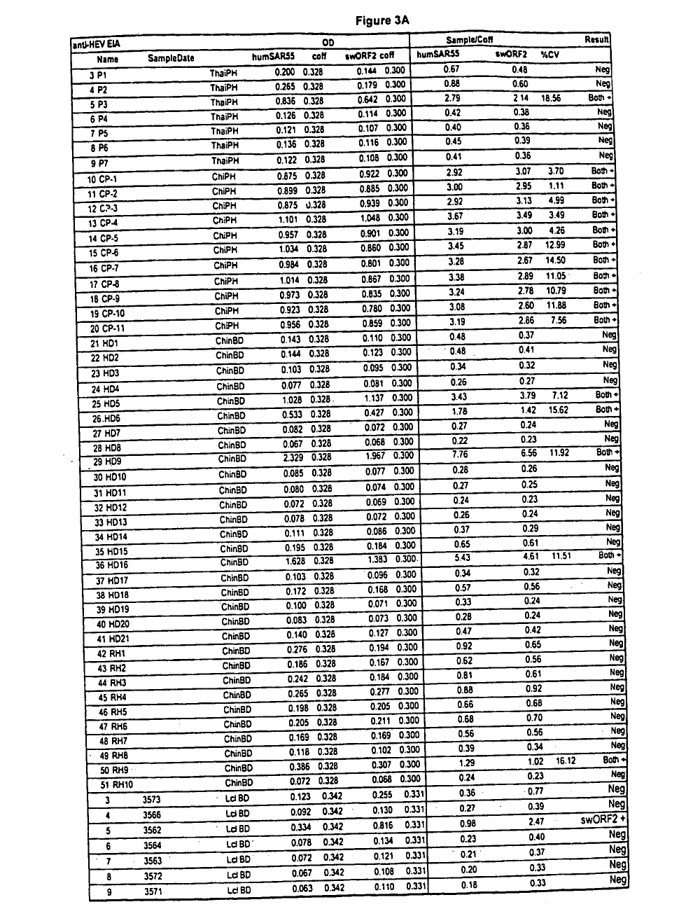

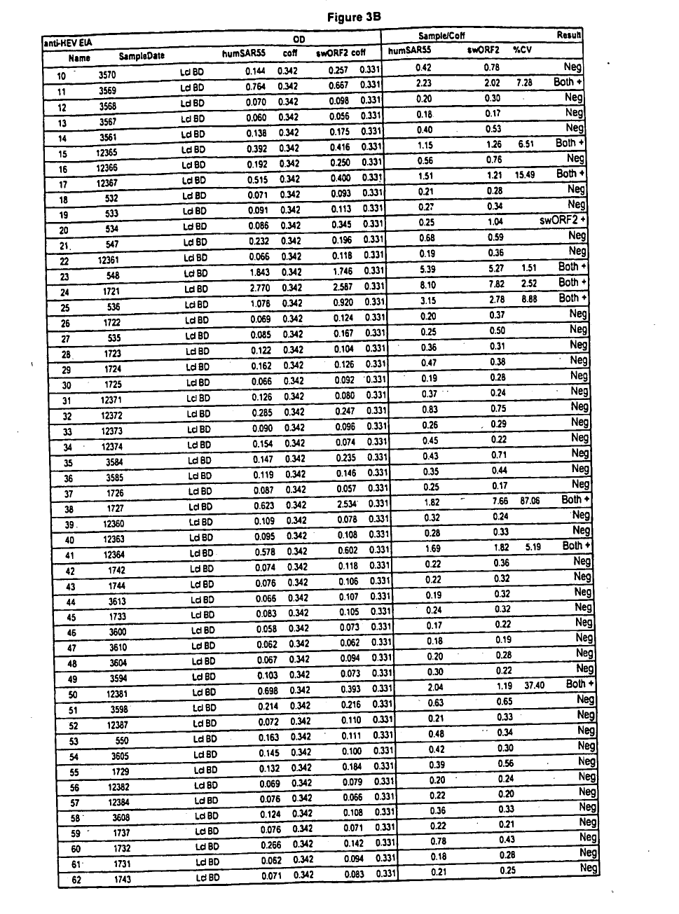

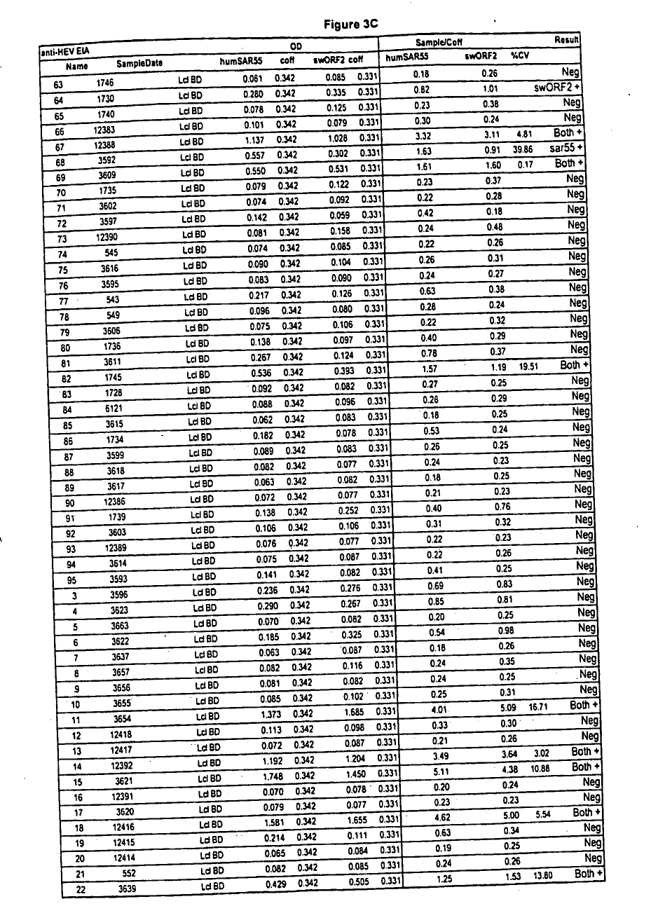

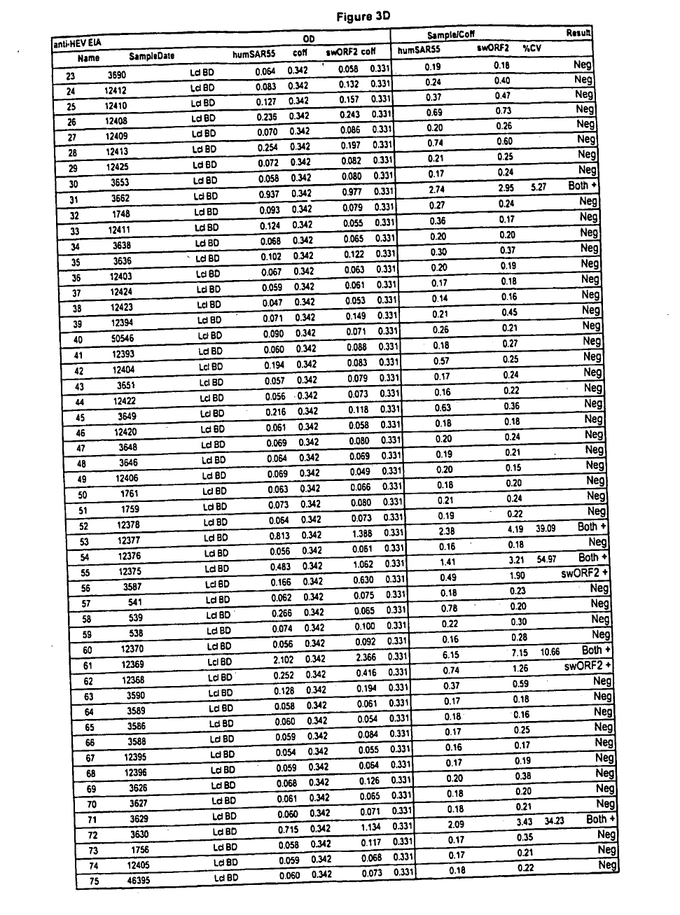

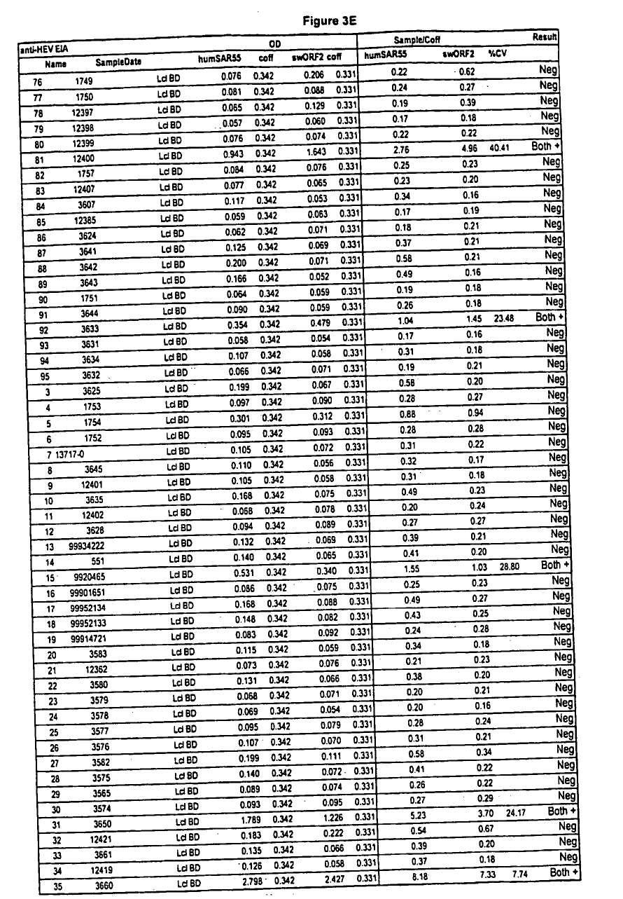

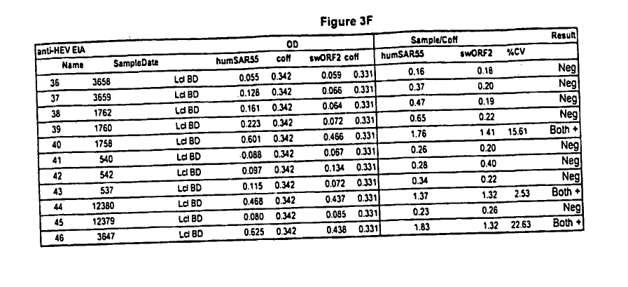

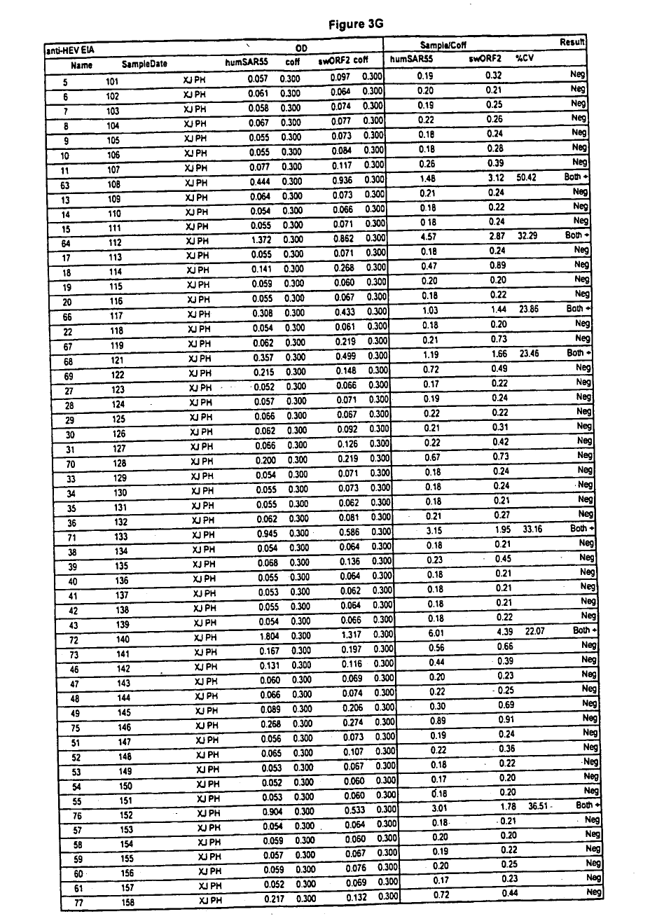

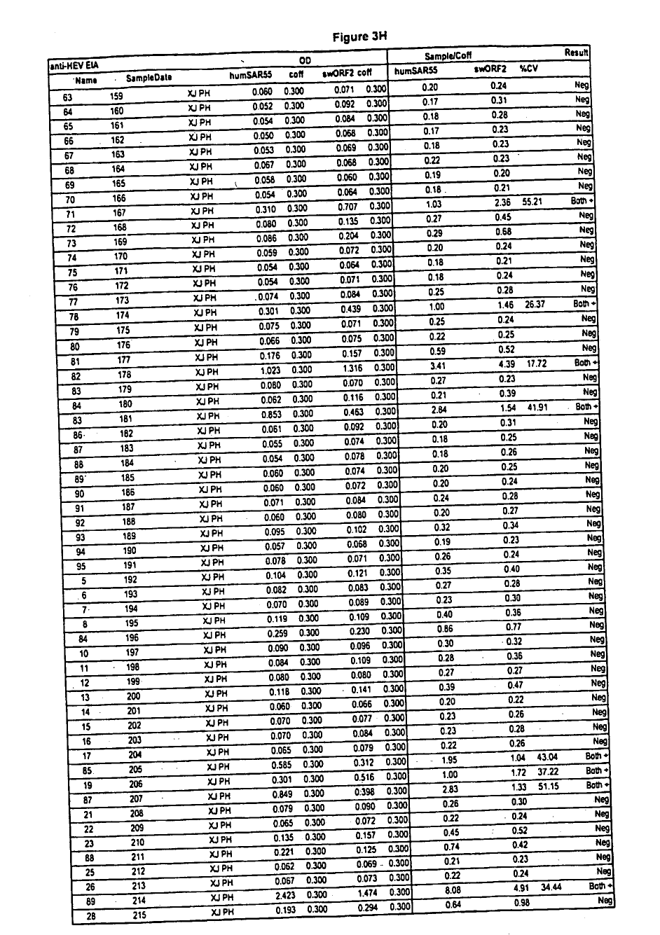

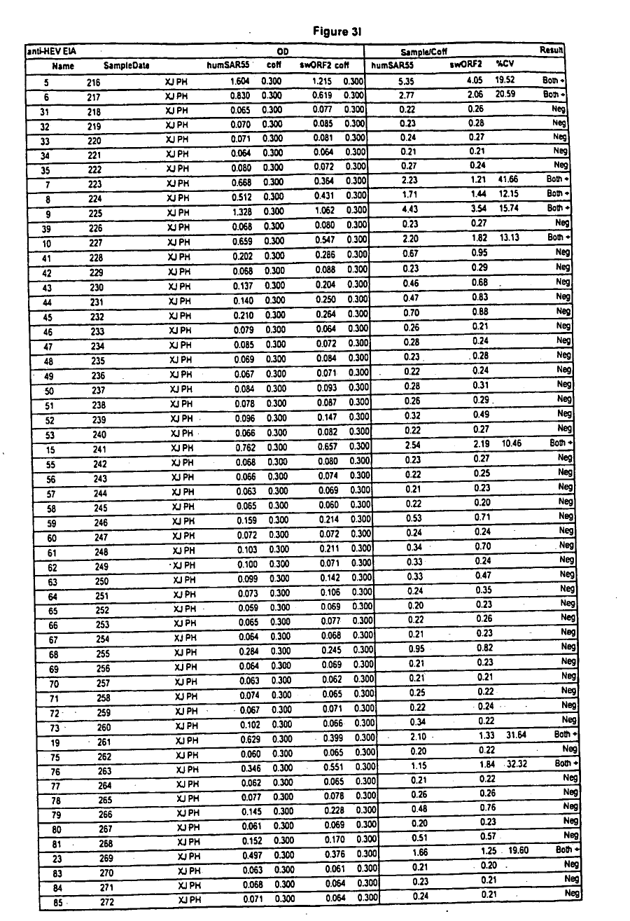

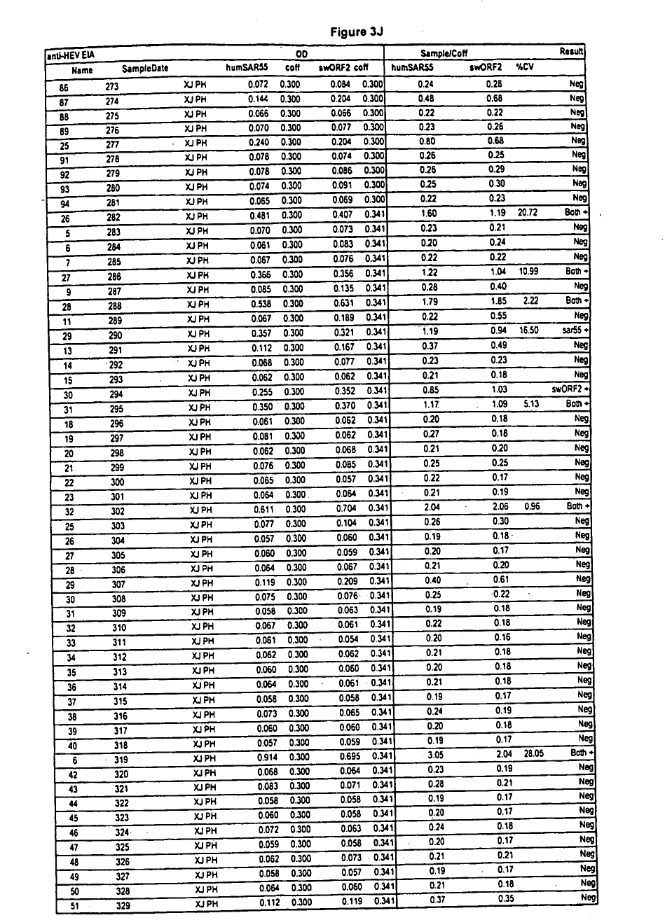

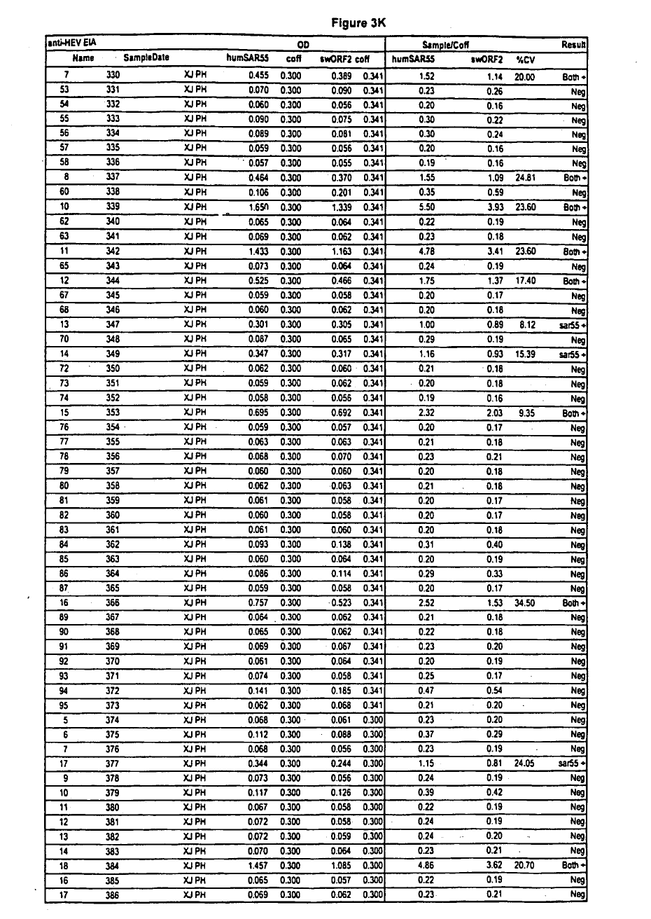

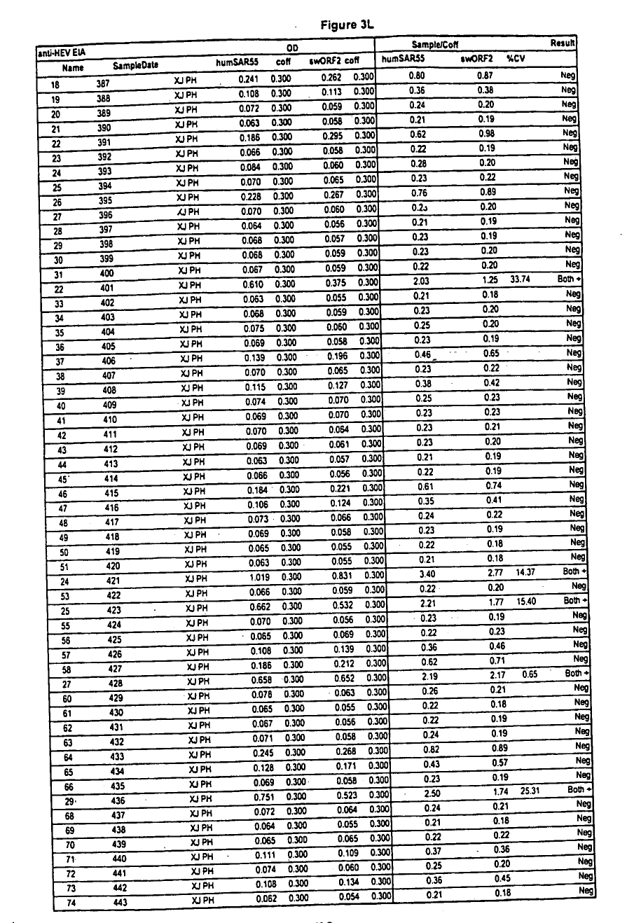

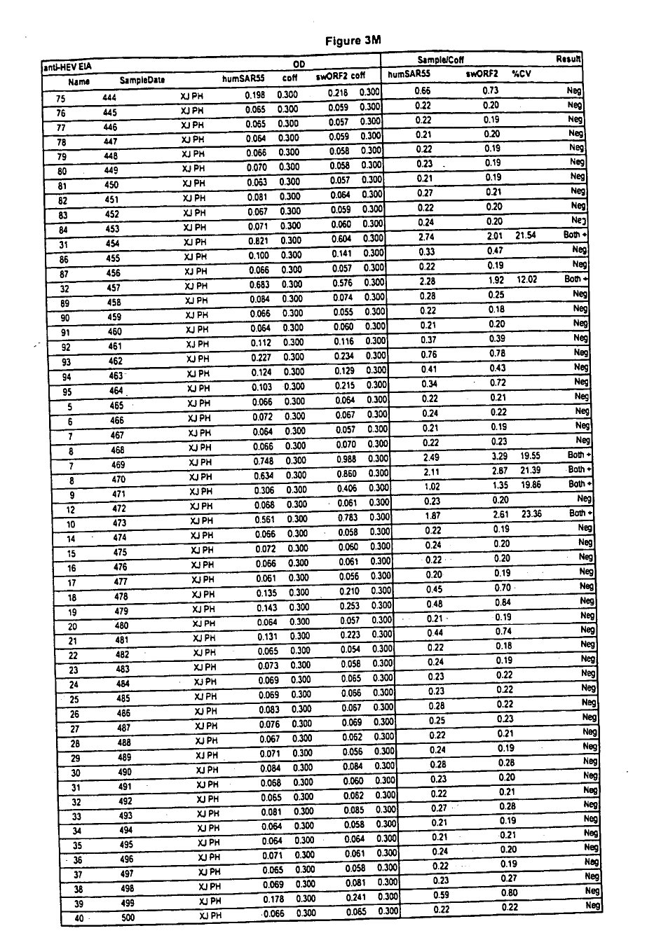

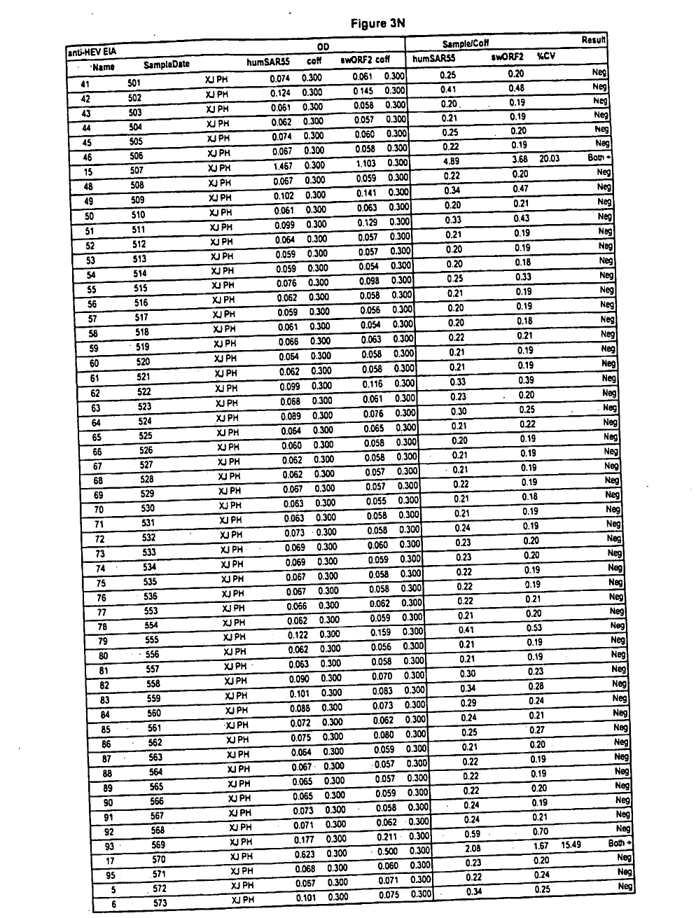

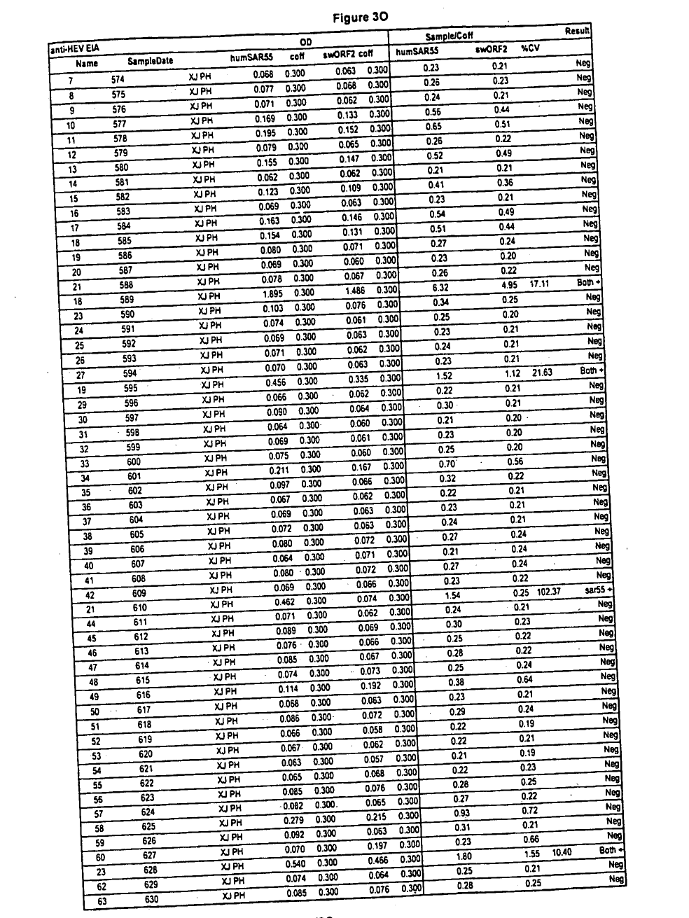

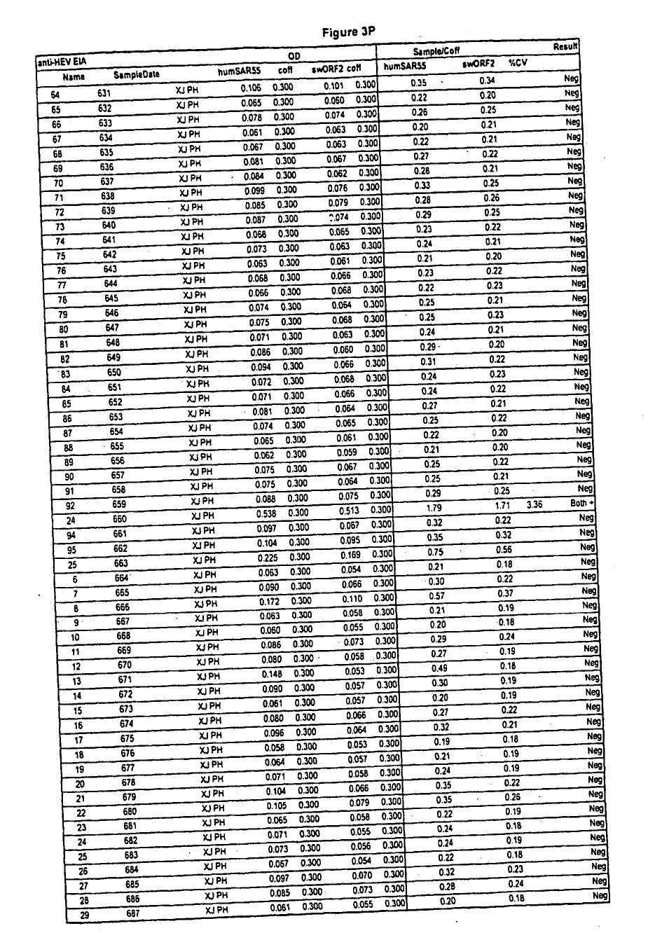

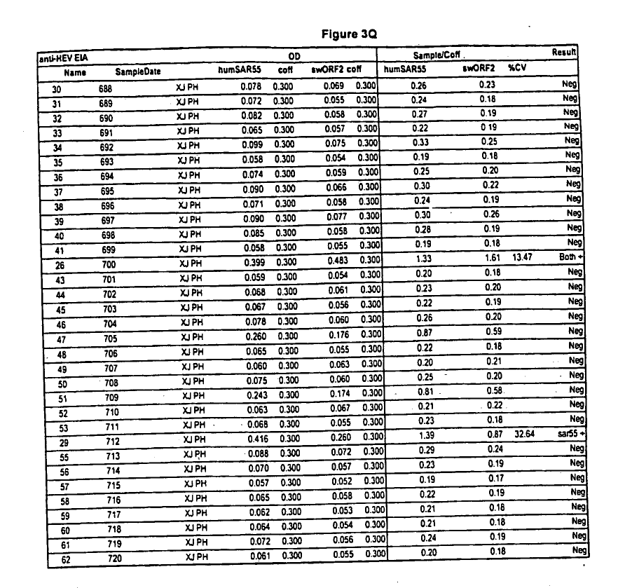

FIGS. 3A-3R show the results of EIAs using as the antigen, either the swine ORF2 protein consisting of amino acids 112-602 of swine ORF2 (designated “swORF2” in the Figures) or the human HEV ORF2 antigen consisting of amino acids 112-607 of the ORF2 of the Pakistani SAR-55 strain of HEV. (designated “humSAR55” in the Figures). Anti-HEV antibody levels were measured in human serum samples. In the Figures the designation “Thai PH” refers to samples from Thai pig handlers, the designation “Chi PH” refers to samples from Chinese pig handlers, the designation “Chin BD” refers to samples from Chinese blood donors, the designation “Lcl BD” refers to samples from US blood donors and the designation “XJPH” refers to samples from US pig handlers. In FIGS. 3A-3Q, a sample was considered positive if the ratio (see column headed “sample/coff”) of the optical density measured for the human SAR55 (“humSAR55” column) or swine antigens (“swORF2” column) to the cutoff value (see columns headed “coff”) for the humSAR55 or swORF2 antigens was greater than 1.0.

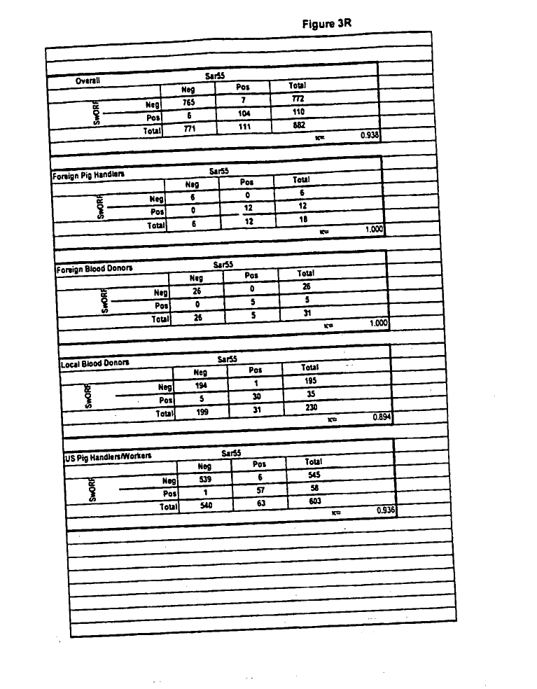

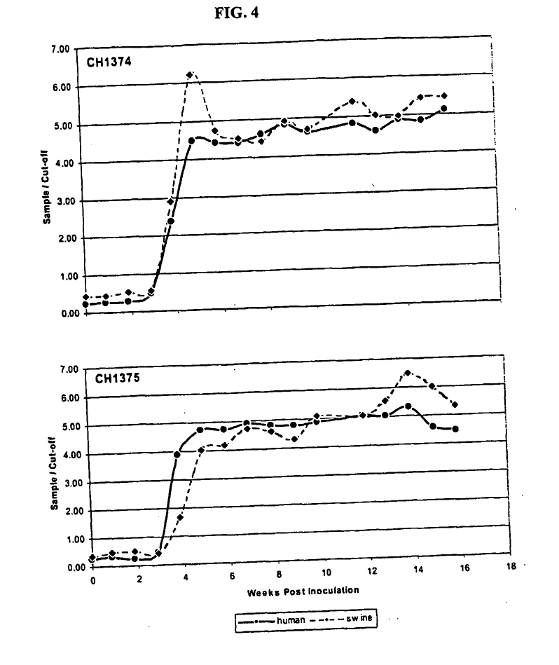

FIG. 4 shows an anti-HEV IgG response time course of two chimpanzees experimentally infected with the Sar-55 strain as determined by EIAs using capsid antigens generated from the human and swine HEV strains. The values are expressed as Sample over Cut-off ratios and 1.0 is the positive baseline.

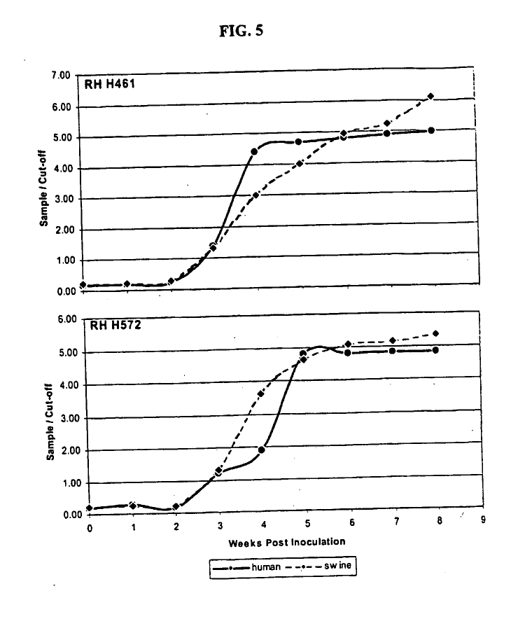

FIG. 5 shows an anti-HEV IgG response time course of two rhesus monkeys experimentally infected with the genotype 2 Mexican strain as determined by EIAs using capsid antigens generated from the Sar-55 and Meng HEV strains.

### DETAILED DESCRIPTION OF INVENTION

The swine hepatitis E virus open reading frame 2 (sHEV ORF2) capsid antigen is structurally very similar to the human HEV ORF2 gene product. Of course, it is not clear whether swine HEV evolved into human HEV, or vice versa, or whether they diverged from a common ancestor. Regardless of lineage, the possibility that swine HEV could infect humans raises a potential public health concern for zoonosis or xenozoonosis, especially since xenotransplantation of pig organs has been suggested as a solution to the solid organ donor shortage for transplantations. Thus, xenozoonoses, the inadvertent transmission of pathogens from animal organs to human recipients, is of major concern. Viruses pathogenic for pigs might pose a risk to humans. However, nonpathogenic pig viruses may also become pathogenic for humans after xenotransplantation, as a result of species-jumping, recombination or adaptation in immunocompromised xenotransplantation recipients. Furthermore, pigs recovered from swine HEV infection might have a damaged liver (or other organ) which would limit usefulness for xenotransplantation.

Because of these and other potential public health concerns, it would be highly advantageous to have a swine HEV ORF2 antigen that is sufficiently closely related to human HEV to allow evaluation as a potential source of infection in humans.

The full-length sHEV ORF2 protein product is predicted to contain 660 amino acids and to weigh 71,000 daltons. Example 3 discloses that expression of the sHEV ORF2 capsid gene from recombinant baculoviruses in insect cells produces multiple HEV capsid polypeptides, including a set of major proteins with molecular weights of 71, 63, and 55 kD. The present invention relates to these proteins and in particular, to the most abundant of these proteins, the 55 kD protein, which is present primarily within the cell by 24 hr. post-infection though a minor fraction of the 55 kD protein is secreted. Amino acid 112 of the full-length sHEV ORF2 is located at the amino terminus of the 55 kD protein as determined by N-terminal sequence analysis. Amino acid 602 of the full-length sHEV ORF2 is located at the carboxy terminus of the 55 kD protein as determined by C-terminal sequence analysis. The present invention therefore relates to nucleic acid molecules which encode this 55 kilodalton swine HEV ORF2 protein. Such nucleic acid molecules can be selected from sequences which encode the swine HEV ORF2 protein sequence shown in FIG. 1A as SEQ. ID NO:1. Preferred nucleic acid sequences are those obtained from the nucleotide sequence of the swine HEV ORF2 shown in FIG. 1B as SEQ. ID NO:2. In one embodiment, the nucleic acid molecule encodes the full-length 660 amino acid ORF2 protein as described in Example 2. Alternatively, the nucleic acid molecule may consist of nucleotides which encode amino acids 112-602 of ORF2 (i.e., nucleotides 334 to 1806 of SEQ. ID NO:2).

Such nucleic acid molecules may be inserted into any vector suitable for expression in prokaryotic or eukaryotic cells. Such vectors include any vectors into which a nucleic acid sequence as described above can be inserted, along with any preferred or required operational elements, and which vector can then be subsequently transferred into a host organism and replicated in such organism. Preferred vectors are those whose restriction sites have been well documented and which contain the operational elements preferred or required for transcription of the nucleic acid sequence.

The “operational elements” as discussed herein include at least one promoter, at least one operator, at least one leader sequence, at least one terminator codon, and any other DNA sequences necessary or preferred for appropriate transcription and subsequent translation of the vector nucleic acid. In particular, it is contemplated that such vectors will contain at least one origin of replication recognized by the host organism along with at least one selectable marker and at least one promoter sequence capable of initiating transcription of the nucleic acid sequence.

In construction of the vector of the present invention, it should additionally be noted that multiple copies of the nucleic acid sequence and its attendant operational elements may be inserted into each vector. In such an embodiment, the host organism would produce greater amounts per vector of the desired HEV protein. The number of multiple copies of the DNA sequence which may be inserted into the vector is limited only by the ability of the resultant vector due to its size, to be transferred into and replicated and transcribed in an appropriate host microorganism.

Preferred expression vectors are those that function in a eukaryotic cell. Examples of such vectors include but are not limited to baculovirus transfer vectors.

The selected recombinant expression vector may then be transfected into a suitable eukaryotic cell system for purposes of expressing the recombinant protein. Preferred cell systems for expression are eukaryotic cells. Such eukaryotic cell systems include, but are not limited to, yeast, insect cells and cell lines such as HeLa, MRC5 or Cv1.

The expressed recombinant protein may be detected by methods known in the art which include SDS-PAGE and Western blotting using sera containing anti-HEV antibody as described in Example 3.

The recombinant protein expressed by the SF9 cells can be obtained as a crude lysate or it can be purified by standard protein purification procedures known in the art which may include differential precipitation, molecular sieve chromatography, ion-exchange chromatography, isoelectric focusing, gel electrophoresis, affinity, and immunoaffinity chromatography and the like. In the case of immunoaffinity chromatography, the recombinant protein may be purified by passage through a column containing a resin which has bound thereto antibodies specific for the ORF protein. An example of a protocol for the purification of the recombinantly expressed 55 kilodalton swine HEV ORF protein is provided in Example 4.

In another embodiment, the expressed recombinant proteins of this invention can be used in immunoassays for the diagnosis or prognosis of hepatitis E in a mammal including, but not limited to, swine and humans. Such assays could be used for detection of swine HEV or similar virus infection in humans, for monitoring pig herds in general, and for risk assessment of swine HEV infection in xenotransplantation using pig organs. In a preferred embodiment, the immunoassay is useful in diagnosing infection of humans and swine with swine hepatitis E. Immunoassays using the swine HEV proteins of the invention therefore provide a highly specific reproducible method for diagnosing swine HEV infections.

Immunoassays of the present invention may be a radioimmunoassay, Western blot assay, immunofluorescent assay, enzyme immunoassay, chemiluminescent assay, immunohistochemical assay and the like. Standard techniques known in the art for EIA are described in Methods in Immunodiagnosis, 2nd Edition, Rose and Bigazzi, eds., John Wiley and Sons, 1980 and Campbell et al., Methods of Immunology, W. A. Benjamin, Inc., 1964, both of which are incorporated herein by reference. Such assays may be a direct, indirect, competitive, or noncompetitive immunoassay as described in the art. (Oellerich, M. 1984. J.Clin. Chem. Clin. BioChem. 22: 895904) Biological samples appropriate for such detection assays include, but are not limited to, tissue biopsy extracts, whole blood, plasma, serum, cerebrospinal fluid, pleural fluid, urine and the like.

In one embodiment, test serum is reacted with a solid phase reagent having surface-bound recombinant swine HEV ORF2 protein as an antigen, preferably, the HEV protein is the swine ORF2 protein consisting of amino acids 112-602 of SEQ. ID NO:1. The solid surface reagent can be prepared by known techniques for attaching protein to solid support material. These attachment methods include nonspecific adsorption of the protein to the support or covalent attachment of the protein to a reactive group on the support. After reaction of the antigen with anti-HEV antibody, unbound serum components are removed by washing and the antigen-antibody complex is reacted with a secondary antibody such as labelled antihuman antibody. The label may be an enzyme which is detected by incubating the solid support in the presence of a suitable fluorimetric or colorimetric reagent. Other detectable labels may also be used, such as radiolabels or colloidal gold, and the like.

In one embodiment, protein expressed by a recombinant baculovirus vector containing the entire ORF2 sequence of swine HEV is used as a specific binding agent to detect anti-HEV antibodies, preferably IgG or IgM antibodies. FIGS. 2 and 3 show the results of EIAs in which the solid phase reagent has the recombinant swine ORF2 protein consisting of amino acids 112-602 as the surface antigen.

The HEV protein and analogs may be prepared in the form of a kit, alone, or in combinations with other reagents such as secondary antibodies, for use in immunoassays.

The recombinant HEV proteins can be used as a vaccine to protect mammals against challenge with hepatitis E derived from human, swine or other species. The vaccine, which acts as an immunogen, may be a cell, cell lysate from cells transfected with a recombinant expression vector or a culture supernatant containing the expressed protein. Alternatively, the immunogen is a partially or substantially purified recombinant protein. While it is possible for the immunogen to be administered in a pure or substantially pure form, it is preferable to present it as a pharmaceutical composition, formulation or preparation.

The formulations of the present invention, both for veterinary and for human use, comprise an immunogen as described above, together with one or more pharmaceutically acceptable carriers and optionally other therapeutic ingredients. The carrier(s) must be “acceptable” in the sense of being compatible with the other ingredients of the formulation and not deleterious to the recipient thereof. The formulations may conveniently be presented in unit dosage form and may be prepared by any method well-known in the pharmaceutical art.

All methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product into the desired formulation.

Formulations suitable for intravenous intramuscular, subcutaneous, or intraperitoneal administration conveniently comprise sterile aqueous solutions of the active ingredient with solutions which are preferably isotonic with the blood of the recipient. Such formulations may be conveniently prepared by dissolving solid active ingredient in water containing physiologically compatible substances such as sodium chloride (e.g. 0.1-2.0M), glycine, and the like, and having a buffered pH compatible with physiological conditions to produce an aqueous solution, and rendering said solution sterile. These may be present in unit or multidose containers, for example, sealed ampoules or vials.

The formulations of the present invention may incorporate a stabilizer. Illustrative stabilizers are polyethylene glycol, proteins, saccharides, amino acids, inorganic acids, and organic acids which may be used either on their own or as admixtures. These stabilizers are preferably incorporated in an amount of 0.1 to 1:10,000 parts by weight per part by weight of immunogen. If two or more stabilizers are to be used, their total amount is preferably within the range specified above. These stabilizers are used in aqueous solutions at the appropriate concentration and pH. The specific osmotic pressure of such aqueous solutions is generally in the range of 0.1-3.0 osmoles, preferably in the range of 0.8-1.2. The pH of the aqueous solution is adjusted to be within the range of 5.0-9.0, preferably within the range of 6-8. In formulating the immunogen of the present invention, an anti-adsorption agent may be used.

Additional pharmaceutical methods may be employed to control the duration of action. Controlled release preparations may be achieved through the use of polymer to complex or absorb the proteins or their derivatives. The controlled delivery may be exercised by selecting appropriate macromolecules (for example polyester, polyamino acids, polyvinyl, pyrrolidone, ethylenevinylacetate, methylcellulose, carboxymethylcellulose, or protamine sulfate) and the concentration of macromolecules as well as the methods of incorporation in order to control release. Another possible method to control the duration of action by controlled release preparations is to incorporate the proteins, protein analogs or their functional derivatives, into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatin microcapsules and poly(methylmethacylate) microcapsules, respectively, or in colloidal drug delivery systems, for example, liposomes, albumin microspheres, microemulsions, nanoparticles, and nanocapsules or in macroemulsions.

When oral preparations are desired, the compositions may be combined with typical carriers, such as lactose, sucrose, starch, talc magnesium stearate, crystalline cellulose, methyl cellulose, carboxymethyl cellulose, glycerin, sodium alginate or gum arabic among others.

The proteins of the present invention may be supplied in the form of a kit, alone, or in the form of a pharmaceutical composition as described above.

Vaccination can be conducted by conventional methods. For example, the immunogen can be used in a suitable diluent such as saline or water, or complete or incomplete adjuvants. Further, the immunogen may or may not be bound to a carrier to make the protein immunogenic. Examples of such carrier molecules include but are not limited to bovine serum albumin (BSA), keyhole limpet hemocyanin (KLH), tetanus toxoid, and the like. The immunogen can be administered by any route appropriate for antibody production such as intravenous, intraperitoneal, intramuscular, subcutaneous, and the like. The immunogen may be administered once or at periodic intervals until a significant titer of anti-HEV antibody is produced. The antibody may be detected in the serum using an immunoassay.

In yet another embodiment, the immunogen may be nucleic acid sequence capable of directing host organism synthesis of an HEV ORF protein. Such nucleic acid sequence may be inserted into a suitable expression vector by methods known to those skilled in the art. Expression vectors suitable for producing high efficiency gene transfer in vivo include, but are not limited to, retroviral, adenoviral and vaccinia viral vectors. Operational elements of such expression vectors are disclosed previously in the present specification and are known to one skilled in the art. Such expression vectors can be administered intravenously, intramuscularly, subcutaneously, intraperitoneally or orally.

In an alternative embodiment, direct gene transfer may be accomplished via intramuscular injection of, for example, plasmid-based eukaryotic expression vectors containing a nucleic acid sequence capable of directing host organism synthesis of HEV ORF protein(s). Such an approach has previously been utilized to produce the hepatitis B surface antigen In vivo and resulted in an antibody response to the surface antigen (Davis, H. L. et al. (1993) Human Molecular Genetics, 2:1847-1851; see also Davis et al. (1993) Human Gene Therapy, 4:151-159 and 733-740).

When the immunogen is a partially or substantially purified recombinant swine HEV ORF2 protein, dosages effective to elicit a protective antibody response against HEV range from about 0.5 μg to about 50 μg. A more preferred range is from about 1 μg to about 30 μg and a most preferred range is from about 5 μg to about 20 μg.

Dosages of swine HEV ORF2 protein-encoding nucleic acid sequence effective to elicit a protective antibody response against HEV range from about 1 to about 5000 μg; a more preferred range being about 300 to about 1000 μg.

The expression vectors containing a nucleic acid sequence capable of directing host organism synthesis of a swine HEV ORF2 protein(s) may be supplied in the form of a kit, alone, or in the form of a pharmaceutical composition as described above.

The administration of the immunogen of the present invention may be for either a prophylactic or therapeutic purpose. When provided prophylactically, the immunogen is provided in advance of any exposure to HEV or in advance of any symptom due to HEV infection. The prophylactic administration of the immunogen serves to prevent or attenuate any subsequent infection of HEV in a mammal. When provided therapeutically, the immunogen is provided at (or shortly after) the onset of the infection or at the onset of any symptom of infection or disease caused by HEV. The therapeutic administration of the immunogen serves to attenuate the infection or disease.

A preferred embodiment is a vaccine prepared using the recombinant swine ORF2 protein expressed by the ORF2 sequence of swine HEV encoding amino acids 1-660 of ORF2. Since the recombinant swine ORF2 protein (112-602) has already been demonstrated to be reactive with a variety of HEV-positive sera from swine and humans (FIGS. 2 and 3), its utility in protecting against HEV strains is indicated.

In addition to use as a vaccine, the compositions can be used to prepare antibodies. The antibodies can be used directly as antiviral agents. To prepare antibodies, a host animal is immunized using the virus particles or, as appropriate, nonparticle antigens native to the virus particle can be administered in conjunction with an adjuvant as described above for vaccines. The host serum or plasma is collected following an appropriate time interval to provide a composition comprising antibodies reactive with the virus particle. The gamma globulin fraction or the IgG antibodies can be obtained, for example, by use of saturated ammonium sulfate or DEAE Sephadex, or other techniques known to those skilled in the art. The antibodies are substantially free of many of the adverse side effects which may be associated with other antiviral agents such as drugs.

The antibody compositions can be made even more compatible with the host system by minimizing potential adverse immune system responses. This is accomplished by removing all or a portion of the Fc portion of a foreign species antibody or using an antibody of the same species as the host animal, for example, the use of antibodies from human/human hybridomas. Humanized antibodies (i.e., non-immunogenic in a human) may be produced, for example, by replacing an immunogenic portion of an antibody with a corresponding, but non-immunogenic portion (i.e., chimeric antibodies). Such chimeric antibodies may contain the reactive or antigen binding portion of an antibody from one species and the Fc portion of an antibody (non-immunogenic) from a different species. Examples of chimeric antibodies, include but are not limited to, non-human mammal-human chimeras, rodent-human chimeras, murine-human and rat-human chimeras (Robinson et al., International Patent Application 184,187; Taniguchi M., European Patent Application 171,496; Morrison et al., European Patent Application 173,494; Neuberger et al., PCT Application WO 86/01533; Cabilly et al., (1987) Proc. Natl. Acad. Sci. USA 84:3439; Nishimura et al., (1987) Canc. Res. 47:999; Wood et al., (1985) Nature 314:446; Shaw et al., (1988) J. Natl. Cancer Inst. 80: 15553, all incorporated herein by reference).

General reviews of “humanized” chimeric antibodies are provided by Morrison S., (1985) Science 229:1202 and by Oi et al., (1986) BioTechniques 4:214.

Suitable “humanized” antibodies can be alternatively produced by CDR or CEA substitution (Jones et al., (1986) Nature 321:552; Verhoeyan et al., (1988) Science 239:1534; Biedleret al. (1988) J. Immunol. 141:4053, all incorporated herein by reference).

The antibodies or antigen binding fragments may also be produced by genetic engineering. The technology for expression of both heavy and light chain genes in E. coli is the subject the PCT patent applications; publication number WO 901443, WO 901443, and WO 9014424 and in Huse et al., (1989) Science 246:12751281.

The antibodies can also be used as a means of enhancing the immune response. The antibodies can be administered in amounts similar to those used for other therapeutic administrations of antibody. For example, pooled gamma globulin is administered at 0.02-0.1 ml/lb body weight during the early incubation period of other viral diseases such as rabies, measles and hepatitis B to interfere with viral entry into cells. Thus, antibodies reactive with the HEV virus particle can be passively administered alone or in conjunction with another antiviral agent to a host infected with an HEV to enhance the effectiveness of an antiviral drug.

Alternatively, anti-HEV antibodies can be induced by administering anti-idiotype antibodies as immunogens. Conveniently, a purified anti-HEV antibody preparation prepared as described above is used to induce anti-idiotype antibody in a host animal. The composition is administered to the host animal in a suitable diluent. Following administration, usually repeated administration, the host produces anti-idiotype antibody. To eliminate an immunogenic response to the Fc region, antibodies produced by the same species as the host animal can be used or the FC region of the administered antibodies can be removed. Following induction of anti-idiotype antibody in the host animal, serum or plasma is removed to provide an antibody composition. The composition can be purified as described above for anti-HEV antibodies, or by affinity chromatography using anti-HEV antibodies bound to the affinity matrix. The anti-idiotype antibodies produced are similar in conformation to the authentic HEV antigen and may be used to prepare an HEV vaccine rather than using an HEV particle antigen.

When used as a means of inducing anti-HEV virus antibodies in an animal, the manner of injecting the antibody is the same as for vaccination purposes, namely intramuscularly, intraperitoneally, subcutaneously or the like in an effective concentration in a physiologically suitable diluent with or without adjuvant. One or more booster injections may be desirable.

The HEV-derived proteins of the invention are also intended for use in producing antiserum designed for pre or post-exposure prophylaxis. Here an HEV protein, or mixture of proteins is formulated with a suitable adjuvant and administered by injection to human volunteers, according to known methods for producing human antisera. Antibody response to the injected proteins is monitored, during a several-week period following immunization, by periodic serum sampling to detect the presence of anti-HEV serum antibodies, using an immunoassay as described herein.

The antiserum from immunized individuals may be administered as a pre-exposure prophylactic measure for individuals who are at risk of contracting infection. The antiserum is also useful in treating an individual post-exposure, analogous to the use of high titer antiserum against hepatitis B virus for post-exposure prophylaxis. Of course, those of skill in the art would readily understand that immune globulin (HEV immune globulin) purified from the antiserum of immunized individuals using standard techniques may be used as a pre-exposure prophylactic measure or in treating individuals post-exposure.

For both in vivo use of antibodies to HEV virus-like particles and proteins and anti-idiotype antibodies and diagnostic use, it may be preferable to use monoclonal antibodies. Monoclonal anti-virus particle antibodies or anti-idiotype antibodies can be produced as follows. The spleen or lymphocytes from an immunized animal are removed and immortalized or used to prepare hybridomas by methods known to those skilled in the art. (Goding, J. W. 1983. Monoclonal Antibodies: Principles and Practice, Pladermic Press, Inc., NY, N.Y., pp. 5697). To produce a human-human hybridoma, a human lymphocyte donor is selected. A donor known to be infected with HEV (where infection has been shown for example by the presence of anti-virus antibodies in the blood or by virus culture) may serve as a suitable lymphocyte donor. Lymphocytes can be isolated from a peripheral blood sample or spleen cells may be used if the donor is subject to splenectomy. EpsteinBarr virus (EBV) can be used to immortalize human lymphocytes or a human fusion partner can be used to produce humanhuman hybridomas. Primary in vitro immunization with peptides can also be used in the generation of human monoclonal antibodies.

Antibodies secreted by the immortalized cells are screened to determine the clones that secrete antibodies of the desired specificity. For monoclonal anti-virus particle antibodies, the antibodies must bind to HEV virus particles. For monoclonal anti-idiotype antibodies, the antibodies must bind to anti-virus particle antibodies. Cells producing antibodies of the desired specificity are selected.

In another embodiment, antibody phage display libraries can be constructed from variable heavy and light chain antibody genes using a phage display vector specifically designed for the expression of antibody fragments to an antigen (Winter et al., (1994) Annu. Rev. Immunol. 12:433-55; de Kruif et al., (1996) Immunol. Today 17: 453-5; Burton et al., (1994) Science 266:1024-7). From such libraries, large numbers of monoclonal antibodies to an antigen of choice can be cloned and isolated. The technique produces high affinity monoclonal antibodies for use in passive immunoprophylaxis.

The above described antibodies and antigen binding fragments thereof may be supplied in kit form alone, or as a pharmaceutical composition for in vivo use. The antibodies may be used for therapeutic uses, diagnostic use in immunoassays or as an immunoaffinity agent to purify ORF 2 proteins as described herein.

### EXAMPLES

### Example 1

### Baculovirus Cloning of Swine HEV ORF2 Gene

A PCR DNA fragment containing a full-length copy of sHEV ORF2 cDNA was digested with the restriction endonucleases Bam HI and Xho 1. The digestion products were purified on a QIA quick column and ligated into the respective sites of the bacterial TA-cloning vector pCR2. 1. The ligation products were used to transform competent E. coli DH5α cells, and bacterial clones containing plasmids with the sHEV ORF2 gene insert were selected by DNA gel analysis of miniprep plasmid DNA. Plasmid DNA of bacterial clone pCRsHEV-9 was digested with Bam HI and Xho I. A 1992 bp DNA fragment was isolated from the restricted DNA and ligated into the bacmid transfer vector pFASTBAC-1 at the Bam HI and Xho I sites located downstream of the baculovirus polyhedrin promoter. The ligation products were used to transform competent E. coli DH5α cells, and bacterial clones containing plasmids with the sHEV ORF2 gene were selected by DNA gel analysis of miniprep plasmid DNA. Digestion of plasmid DNA from the bacterial clone designated pFBsHEV ORF2 (6,681 bp) with Bam HI and Xho I released a 1992 bp DNA fragment as expected for the sHEV ORF2 DNA insert.

pFBsHEV ORF2 DNA was transformed into competent E. coli DHIOBac cells containing parental bacmid DNA to facilitate site-specific recombination of the sHEV ORF2 gene into the baculovirus genome within the polh locus. Recombinant bacmid DNA was isolated from amplified bacterial cultures derived from white antibiotic resistant colonies. Bacmid DNA containing sHEV ORF2 DNA was transfected into Sf-9 cells using the cationic lipid CELLFECTIN. Transfected cells were harvested after three days and assayed for expression of sHEV ORF2 capsid proteins by SDS-PAGE and Western blot analysis using antisera to human HEV. A single protein band with a molecular weight of 55,000 daltons was detected in the transfected cells by immunoblotting with the anti-HEV sera. Recombinant baculoviruses in culture media from transfected cells harvested at 72 hours post-transfection was used to infect Sf-9 insect cells in agarose plaque assays. Virus from plaques was isolated and amplified further in Sf-9 insect cells. The resulting recombinant baculovirus expressed sHEV ORF2 proteins in Sf-9 insect cells.

### Example 2

### Establishment of Master Virus Seed Bank

A virus stock designated bsHEV ORF2 (R257) was prepared in Sf-9 cells following three serial plaque purifications. No wild type baculovirus was present in the virus stock as demonstrated by the absence of wild-type plaque morphology and β-galactosidase expression in agarose plaque assays. Baculovirus genomic DNA was isolated from recombinant virus in the virus stock and subjected to nucleotide sequence analysis using the cycle sequencing technique. The location of the swine HEV ORF2 DNA insert (1992 bp) was confirmed to be in-frame and downstream of the polyhedrin promoter in the polh locus as expected. The observed nucleotide sequence shared 100% homology with the nucleotide sequence of the swine HEV ORF2 shown in FIG. 1. This bsHEV ORF2 baculovirus stock was tested for microbial sterility, mycoplasma and spiroplasma contamination, and the presence of endotoxins. No microbial contaminants were detected by these tests, and an endotoxin level of 0.1 EU/ml was observed. bsHEV ORF2 (R257) was designated as the master virus seed bank and stored in 10 ml aliquots at 2° C., −8° C., and −70° C. The virus titer of R257 was 2.9×10⁷ pfu/ml as determined by agarose plaque assay using Sf-9 cells.

### Example 3

### Expression of Recombinant Swine HEV ORF2 Proteins in Insect Cells

Temporal expression of the swine HEV ORF2 gene in baculovirus-infected cells was investigated. Sf-9 insect cells cultivated as shaker suspension cultures in serum-free medium were infected with recombinant baculoviruses encoding the full-length swine hepatitis E virus ORF2 gene. Cell lysates and media were harvested from virus infections daily for four consecutive days and analyzed by SDS-PAGE and immunoblotting methods.

The results showed that in addition to the full-length ORF2 product of 71 kD, multiple sHEV related proteins appeared in infected cells and in the media. The most abundant of these proteins had a molecular weight of 55 kD. The HEV 71 kD protein was detected as early as one day post-infection in infected cell lysates and media and accumulated for several more days in cells but disappeared in media by four days post-infection. Another sHEV protein (˜63 kD) appeared in infected cells and media by one day post-infection and accumulated over the next two days. At four days post-infection, the level of 63 kD protein in cells and media decreased. A shEV 55 kD protein appeared in cells and in media by two days post-infection. The sHEV 55 kD protein accumulated intracellularly at days three and four post-infection. Additionally, sHEV proteins with other molecular weights, but in smaller amounts, were observed intracellularly and extracellularly.

### Example 4

### Recombinant sHEV ORF2 Protein Purification.

Recombinant sHEV ORF2 proteins were purified from Sf-9 insect cell cultures infected with recombinant baculoviruses expressing the full-length sHEV ORF2 gene using a purification scheme that included anion exchange and size exclusion chromatography. Recombinant swine HEV ORF2 proteins were purified from clarified baculovirus-infected cell lysates. Cell lysates were prepared at 4° C. for 30 minutes by differential lysis of infected cells harvested at five days post-infection with the nonionic detergent, Nonidet P-40, at a final concentration of 0.5%. Following cell lysis and removal of infected cell nuclei by centrifugation, cell lysates were diluted 1:10 with Q loading buffer (50 mM Tris-HCl, pH 8.0, 10 mM NaCl) to reduce the ionic strength. In contrast, media harvested from virus infections were clarified by centrifugation, concentrated 10 fold by tangential flow ultrafiltration using hollow fiber filters comprised of polysulfone, and subjected to diafiltration against Q loading buffer to reduce the ionic strength.

Recombinant sHEV ORF2 proteins in cell lysates and media were captured by anion exchange chromatography. Diluted crude lysate (1.5 bed vol.) was loaded onto a Q Sepharose Fast Flow strong anion exchange column (XK50 column, 5.0×7.5 cm, 150 ml; Pharmacia, Piscataway, N.J.) at a flow rate of 10.0 ml/min. The column was washed first with 1.0 bed volume of loading buffer at a flow rate of 10 ml/min. followed by a second wash with 1.0 bed volume of loading buffer at a flow rate of 20 ml/min. Proteins were eluted with 7.5 bed volumes of a continuous linear NaCl gradient (10-300 mM) in loading buffer at a flow rate of 20 ml/min. Recombinant sHEV ORF2 proteins bound to Q Sepharose Fast Flow resin, a strong anion exchange chromatographic matrix, and selectively eluted at a NaCl concentration of 140 mM as determined by SDS-PAGE and immunoblot analyses of unbound and bound column fractions. Fractions containing sHEV ORF2 55 kD proteins were pooled and desalted by gel filtration through a Sephacryl G-25 column (Pharmacia) with Q loading buffer.

The peak protein fraction from the Sephacryl G-25 column was collected and loaded onto a Source 15 Q High Performance (Pharmacia) strong anion exchange column to resolve and concentrate sHEV ORF2 polypeptides. The Source 15 Q HP column was washed and eluted as described above for anion exchange chromatography using Q Sepharose. Recombinant sHEV ORF2 55 kD proteins bound to the matrix and eluted again at 140 mM NaCl. Peak fractions containing sHEV ORF2 proteins were pooled and fractionated further by size exclusion chromatography using a Superdex G-75 column. Size exclusion chromatography using phosphate-buffered saline (pH 7.2) as a final purification step resolved the recombinant sHEV ORF2 55 kD protein from other protein contaminants as determined by SDS-PAGE and Western blot analyses. The purity of the final bulk product by size exclusion chromatography was >98% as determined by laser scanning densitometry of Coomassie Blue stained gels.

### Example 5

### Amino Terminal Sequence Analysis of sHEV 55 kD Protein.

The amino terminus of the recombinant sHEV ORF2 55 kD protein was determined by automated micro Edman degradation. 11 cycles of direct Edman degradation were performed on the recombinant sHEV ORF2 55 kD proteins. The amino acid sequence corresponded to residues 112 through 122 (AVSPAPDTAPV) of the full-length recombinant sHEV ORF2 gene product. The carboxy terminus of the recombinant sHEV ORF2 55 kD protein was determined by automated chemical cleavage. Three rounds of chemical lysis were performed on recombinant sHEV ORF2 55 kD protein. The amino acid sequence corresponded to residues 600 through 602 (VLA) of the full-length recombinant sHEV ORF2 gene product.

The recombinant swine and human HEV ORF2 proteins produced in baculovirus-infected insect cells share 91.4% protein sequence homology. Both swine and human HEV ORF2 gene products undergo proteolytic cleavage to produce final intracellular products of 55 and 56 kD respectively. The amino termini of these two proteins are similar, as N-terminal cleavages occur between amino acids 111 and 112 of both proteins to produce the final protein products. The C-termini of these proteins differ slightly following C-terminal proteolysis, as the swine HEV ORF2 protein ends at amino acid 602 whereas the human HEV ORF2 protein terminates at amino acid 607.

### Example 6

### Detection by EIA of anti-HEV Antibodies In Sera From Swine

To determine if the insect cell-derived swine HEV ORF2 antigen 112-602 could detect anti-HEV antibody in sera from swine and humans, EIAs were carried out as follows on sera collected from swine and humans using either the 55 kilodalton swine ORF2 protein (amino acids 112-602) or the 56 kilodalton protein of the SAR55 strain of HEV (amino acids 112-607).

Capture Plate Preparation

The antigen preparation was diluted to approximate by 0.5 μg/ml in carbonate buffer (Carbonate-Bicarbonate capsules, Sigma #C-3041, final 0.05M, pH9.6) and 100 μl of the diluted antigen preparation was added to each of 96 wells of a microtiter plate (Linbro/Titertek, ICN#76-381-04). The plates were then incubated for 18 hours at room temperature, washed twice with 0.02% Tween-20 (KPL #50-63-00) solution, and 120 μl of blocking solution was then added and incubated 1 hour at 37° C., followed by washing five times with 0.02% Tween-20 (K&P #50-63-00) solution.

The plates were now ready for use.

Sample Preparation

In a separate microtiter plate, 10-fold dilutions (10¹ 10², 10³, 10⁴, 10⁵, 10⁶) of the starting sample were made in blocking buffer.

100 μl of dilutions to be tested, starting with the 10² dilution, were added into wells of the capture plate. The plate was incubated at 37° C. for 30 minutes and then washed five times with 0.02% Tween-20 solution.

100 μl of secondary antibody (anti-human-IgG-HRPO, KPL #74-1006 prepared to manufacturer's recommendations using the blocking reagent as diluent) was added to each well, incubated 30 minutes at 37° C., and then washed five times with 0.02% Tween-20 solution.

100 μl of ABTS substrate (ABTS-citric acid-H₂O₂, KPL #50-66-01) was t added to each well, then kept covered for 30 minutes. After 30 minutes had elapsed, 100 μl of stop solution (KPL #50-85-02) were added to each well and optical density was read at 405 nm.

Four five-fold dilutions of a WHO anti-HEV standard preparation (95/584, calibrated to 100 Units/ml) obtained from the National Institute for Biological Standards and Control, Hertfordshire, England, starting at 1:400 (0.25 WHO units), was included in each test plate to establish a sensitivity range and develop a standard line from which relative quantity values were extrapolated.

Commercial Reagents

Washing solution, ready-to-use ABTS, HRPO labeled antibodies and BSA were obtained from Kirkegaard & Perry, 2 Cesna Ct, Gaithersburg, Md. 20879. Other reagents are available from Sigma.

EIA Results

The results for the swine sera are shown in FIGS. 2A-2N and for the human sera in FIGS. 3A-3Q and the data are summarized in FIGS. 2O and 3R respectively.

### Example 7

### Use of the Swine 55 Kilodalton ORF2 Protein as a Vaccine

As described above in Example 6, the swine ORF-2 protein is immunoreactive as it has been shown to react with a variety of sera taken from swine and humans infected with HEV. This provides support for the use of this recombinant protein as a vaccine to protect against HEV strains. Mammals, preferably rhesus monkeys or chimpanzees, are immunized by intramuscular injection with purified or partially purified recombinant swine ORF-2 protein (112-602) in an amount sufficient (0.1 to 100 μg) to stimulate the production of protective antibodies. The immunized mammals are then challenged with a wild-type strain of HEV and protection from challenge may be measured by a variety of assays including, but not limited to, assaying sera of immunized mammals for levels of alanine aminotransferase, (ALT), anti-HEV antibodies or HEV RNA by RT-PCR.

### Example 8

### Hepatitis E Virus (HEV) Capsid Antigen Derived From Virus of Human or Swine is Equally Efficient for Detecting Anti-HEV by Enzyme Immunoassay

The goal of this study was to evaluate and compare a pair of enzyme immunoassays for the detection of antibodies to HEV in human and swine sera. Though we tested only swine and human sera, these results likely apply to other species since it is reported that the ORF2 epitopes are broadly reactive across species and strains (Anderson, D. A. et al., (1999) J Virol Methods 81:131-42; Khudyakov, Y. E. et al., (1999) J Clin Microbiol 372863-71; Meng, J. et al., (2001) Virology 288:203-11). The assays we describe here are virtually the same but for the capture antigen each employs, namely a truncated portion of the ORF2 gene product from a swine strain of HEV and from a human strain of HEV. The human strain is the Pakistani Sar-55 strain (Bryan, J. P. et al., (1994) J Infect Dis 170:517-21, and the swine strain is the US Meng strain (Meng, X. J. et al., (1997) J Clin Microbiol 40:117-22).

Serum Samples

Serial weekly serum samples from two chimpanzees and two rhesus monkeys experimentally infected with HEV were compared with both assays. The chimpanzees were infected with the Pakistani strain (Sar-55) representing genotype 1 and the rhesus monkeys were infected with the Mexican strain of HEV, representing genotype 2.

Another sample set consisted of 792 pig sera (360 samples from US, 152 from Canada, 30 from China, 190 from Korea and 60 from Thailand) and 882 human sera (230 samples from US volunteer blood donors, 603 US pig handlers, 18 Thai animal handlers and 31 blood bank volunteers from China) (Meng, S. J. et al., (1999) J Med Virol 59:297-302). Overall, specimens were obtained in areas where HEV genotypes 1, 3 and possibly 4 predominate (Schlauder, G. G. et al., (2001) J Med Virol 65:282-92). All samples were unlinked from the identity of their donors.

Antigen Preparation and Purification

The putative HEV capsid protein (ORF2) was expressed in insect cells (SF9) from a recombinant baculovirus (Robinson, R. A. et al., (1998) Protein Expr Purif 12:75084; Tsarev, S. A. et al., (1993) J Infect Dis 168:369-78). The 72 kD full-length product was processed in the cells to yield a 63-kD peptide, a 55 or 56-kD peptide, and a 53-kD peptide. The 55 or 56-kD antigen was used in the EIA and was purified by anion-exchange and gel filtration chromatography (Robinson, R. A. et al., (1998) Protein Expr Purif 12:75-84). The products of the human and swine strains contained amino acids 112 to 607 (496 amino acids) and 112 to 602 (491 amino acids), respectively.

EIA for the Detection of Anti-HEV IgG in Swine and Humans.

We used a modification of the EIA described by Tsarev (Tsarev, S. A., (1993) J Infect Dis 168:369-78). Polystyrene microwell plates (ICN 76-381-04, Costa Mesa, Calif.) were incubated with ORF2 antigen diluted in a carbonate-bicarbonate (pH 9.6) buffer for 18 hours at room temperature. The antigen concentration was 0.05 μg/well for the human strain and 0.029 μg/well for the swine strain. The optimal concentrations of capture antigen were established by block titration using a known anti-HEV positive chimpanzee serum and a hyperimmune swine anti-HEV positive serum. The wells were washed twice in an automated plate washer with a commercially available wash solution (Kirkegaard & Perry, Gaithersburg, Md.) containing 0.02% Tween 20 in 0.002M imidazole-buffered saline. The wells were blocked with BSA/gelatin for 1 hour at 37° C. prior to freezing at −20° C. in plastic bags. Immediately before use the blocking buffer was removed and the plates were washed twice with wash buffer as described above.

Ten microliters of each test and control sample were diluted 1:10. The sample was further diluted 1:10 into the antigen-coated test plate (1:100 final test dilution) and incubated for 30 minutes at 37° C. Wells were washed 5 times and 100 μl of horseradish peroxidase (HRPO)-labeled anti-IgG (Kirkegaard & Perry, Gaithersburg, Md.) was added to each well. The HRPO-labeled secondary antibodies were species-specific anti-IgG (heavy and light chain) and were used at a net 1.0 μg/ml. Following a 30 minute incubation at 37° C., unbound conjugate was removed by washing 5 times as described above. Azino-diethylbenzotyazol-sulfonate (ABTS) substrate was added for color development and absorbance (405 nm) was read after 30 minutes.

The cutoff for the EIA using swine antigen was established for each test from internal controls and throughout this study ranged between 0.300 and 0.383 with a median of 0.330 (Meng, X. J. et al., (1997) Proc Natl Acad Sci USA 94:9860-5). The positive cut-off for the EIA using the human Sar-55 antigen was similarly established (Tsarev, S. A., (1993) J Infect Dis 168:369-78) and ranged between 0.300 and 0.342 in this study. Previously tested negative blood bank samples, dilution buffer and pre-inoculation swine sera served as negative controls.

Statistical Analysis.

Calculations to determine concordance and prevalence were carried out using the PC version of S-Plus software as an add-on to Microsoft Excel.

### Results

Development of Anti-HEV in Non-human Primates Following Infection, as Measured by Both Assays.

Serial samples from two chimpanzees experimentally infected with the Sar-55 (genotype 1) HEV strain (FIG. 4) and two rhesus monkeys experimentally infected with the Mexican (genotype 2) HEV strain (FIG. 5) were tested with both EIAs. Very similar values were obtained regardless of whether the capture antigen in the EIA was from Sar-55 (genotype 1) or Meng (genotype 3) strain. The agreement for these two sets of data was 98% (Kappa value=0.952, Cl_{95%} 79-106%). In all four cases, seroconversion was detected at the appropriate time and the patterns of antibody positivity were as expected for a normal infection thus validating each assay.

Seroprevalence of HEV in Human Serum or Plasma Samples as Determined by Both Assays.

Human sera from HEV endemic and non-endemic areas were tested with both EIAs. The overall prevalence of anti-HEV in the human sera was virtually the same regardless of the capture antigen. Prevalence was 13% when evaluated with the human capture antigen versus 12% with the swine capture antigen (Table 1). Furthermore, the prevalence values for each of the sub-groups were practically equal.

TABLE 1
Anti-HEV prevalence in human sera as determined
by human or swine antigen capture EIAs.

No. (%) positive for antibody
reactive with indicated antigen

| | Sar-55 | | | |
|---|---|---|---|---|
| | (Human | | Meng | |
| Source | strain) | | (Swine strain) | |
| Foreign Pig Handlers | 12 | (67) | 12 | (67) |
| US Pig Handlers | 63 | (10) | 58 | (10) |
| Foreign Blood Donors | 5 | (16) | 5 | (16) |
| US Blood Bank Volunteers | 31 | (13) | 35 | (15) |
| Total | 111 | (13) | 110 | (12) |

There was a 99% concordance (Kappa value=0.938, Cl_{95%} 97-99) when data from human sera tested with the human and swine ORF2-coated capture plates were compared (Table 2).

TABLE 2
Contingency table comparing results of testing
human serum with the Sar-55 ORF2 and the
Meng ORF2 capture antigens.

| | Sar-55 ORF2 | | |
|---|---|---|---|
| Meng ORF2 | Negative | Positive | Total |
| Negative | 765 | 7 | 772 |
| Positive | 6 | 104 | 110 |
| Total | 771 | 111 | 882 |

Concordance=99%, calculated by dividing the sum of concordant values by the sum total. Kappa value=0.938, Cl_{95%}=97%-99%

Comparisons between data obtained from the two EIAs for foreign pig handlers and blood donors each showed 100% agreement and comparisons of results for US volunteer blood donors and pig handlers yielded concordance values of 97% (Kappa value=0.894, Cl_{95%} 95-99%) and 99% (Kappa value=0.936, Cl_{95%} 98-100%) respectively. Therefore, both antigens reacted equally with anti-HEV in human sera.

Seroprevalence of HEV in Swine as Determined by Both Assays.

Anti-HEV prevalence in swine sera was also measured by EIAs containing each of the capture antigens. Once again, the results with the two capture antigens agreed. The human and swine ORF2 EIAs yielded 37% and 35% prevalence respectively (Table 3).

TABLE 3
Anti-HEV prevalence (%) in swine sera as determined
by human or swine antigen capture EIAs.

No. (%) positive for antibody
reactive with indicated antigen

| | Sar-55 | | | |
|---|---|---|---|---|
| | (Human | | Meng | |
| Source | strain) | | (Swine strain) | |
| USA | 66 | (18) | 69 | (19) |
| Canada | 95 | (63) | 86 | (57) |
| China | 5 | (17) | 3 | (10) |
| Korea | 97 | (51) | 89 | (47) |
| Thailand | 29 | (48) | 34 | (57) |
| Total | 292 | (37) | 281 | (35) |

As seen in Table 4, comparison of test results for swine sera yielded a concordance value of 93% (Kappa value=0.839, Cl_{95%} 86-92%). Independently, the subgroups that made up the swine serum set yielded concordance values of 96% (Kappa value=0.882, Cl_{95%} 93-98%) for the USA, 86% (Kappa value=0.714, Cl_{95%} 60-81%) for Canada, 91% (Kappa value=0.811, Cl_{95%} 76-90%) for Korea, 92% (Kappa value=0.834, Cl_{95%} 71-97%) for Thailand and 93% (Kappa value=0.714, Cl_{95%} 83-102%) for China.

TABLE 4
Contingency table comparing results of
testing swine serum with the Sar-55 ORF2
and the Meng ORF2 capture antigens.

| | Sar-55 ORF2 | | |
|---|---|---|---|
| Meng ORF2 | Negative | Positive | Total |
| Negative | 476 | 35 | 511 |
| Positive | 24 | 257 | 281 |
| Total | 500 | 292 | 792 |

Concordance=93%. Kappa value=0.839, Cl_{95%}=86%-92%

These data demonstrate the comparable ability of each of the capture antigens to identify anti-HEV in swine serum.

The contents of all citations, i.e., journal articles, patents and the like, are incorporated herein by reference.

It is understood that the examples and embodiments described herein are for illustrative purposes and that various modifications and changes in light thereof to persons skilled in the art are included within the spirit and purview of this application and scope of the appended claims.

1. A swine hepatitis E virus open-reading frame 2 protein consisting of amino acids 112 to 602.

2. A swine hepatitis E virus open-reading frame 2 protein consisting of amino acids 112 to 602 of SEQ ID NO: 1.

3. A pharmaceutical composition comprising the protein of claim 1 and a suitable excipient, diluent or carrier.

4. A pharmaceutical composition comprising the protein of claim 2 and a suitable excipient, diluent or carrier.

5. A method of preventing hepatitis E, comprising administering the pharmaceutical composition of claim 3 to a mammal in an amount sufficient to stimulate the production of protective antibodies.

6. A method of preventing hepatitis E, comprising administering the pharmaceutical composition of claim 4 to a mammal in an amount sufficient to stimulate the production of protective antibodies.

7. A vaccine for immunizing a mammal against hepatitis E, said vaccine comprising a protein according to claim 1 in a pharmaceutically acceptable carrier.

8. A vaccine for immunizing a mammal against hepatitis E, said vaccine comprising a protein according to claim 2 in a pharmaceutically acceptable carrier.

9. A kit for preventing hepatitis E in a mammal, said kit comprising a protein according to claim 1.

10. A kit for preventing hepatitis E in a mammal, said kit comprising a protein according to claim 2.

11. A DNA molecule having a sequence consisting of nucleotides which encode amino acids 112 to 602 of a swine hepatitis E virus open reading frame 2 protein.

12. The DNA molecule of claim 11, wherein the molecule encodes amino acids 112 to 602 of SEQ ID NO:1.

13. A recombinant expression vector comprising a DNA molecule according to claims 11 or 12.

14. A host cell containing an expression vector according to claim 13.

15. A method of producing a recombinant hepatitis E virus open reading frame 2 protein, said method comprising:
(a) culturing a host cell of claim 14 under conditions appropriate to cause expression of said protein; and
(b) obtaining said expressed protein from the host cell.

16. A method of detecting antibodies to hepatitis E virus in a biological sample, said method comprising:
(a) contacting said sample with a swine hepatitis E virus open-reading frame 2 protein consisting of amino acids 112 to 602; and
(b) detecting immune complexes formed between said protein and said antibodies, wherein detection of said complexes indicates the presence of antibodies to hepatitis E virus in said sample.

17. The method of claim 16, wherein the protein consists of amino acids 112-602 of SEQ ID NO:1.

18. A kit for use in a method of detecting antibodies to hepatitis E virus in a biological sample, said kit comprising a swine hepatitis E virus open-reading frame 2 protein consisting of amino acids 112 to 602.

19. The kit of claim 18, wherein the protein consists of amino acids 112-602 of SEQ ID NO:1.

20. Antibodies having specific binding affinity for a swine hepatitis E virus open-reading frame 2 protein consisting of amino acids 112 to 602.

21. The antibodies of claim 16, wherein said antibodies have specific binding affinity for a protein consisting of amino acids 112-602 of SEQ ID NO:1.

22. A method for detecting hepatitis E virus in a biological sample, said method comprising;
(a) contacting said sample with the antibodies of claim 20 to form an immune complex with said hepatitis E virus; and
(b) detecting the presence of said complex, wherein detection of said complex indicates the presence of hepatitis E virus in said sample.

23. A method for detecting hepatitis E virus in a biological sample, said method comprising;
(a) contacting said sample with the antibodies of claim 21 to form an immune complex with said hepatitis E virus; and
(b) detecting the presence of said complex, wherein detection of said complex indicates the presence of hepatitis E virus in said sample.

24. A method for producing the antibodies of claim 20, said method comprising immunizing a mammal with a swine hepatitis E virus open-reading frame 2 protein consisting of amino acids 112 to 602.

25. A method for producing the antibodies of claim 21, said method comprising immunizing a mammal with a protein consisting of amino acids 112-602 of SEQ ID NO:1.

26. A DNA molecule having a sequence consisting of nucleotides which encode amino acids 112-660 of a swine hepatitis E virus open reading frame 2 protein.

27. The DNA molecule of claim 26, wherein the molecule encodes amino acids 112-660 of SEQ ID. NO.1.

28. A recombinant expression vector comprising a DNA molecule according to claims 26 and 27.

29. A host cell containing an expression vector according to claim 28.

30. A method of producing a recombinant hepatitis E virus open reading frame 2 protein, said method comprising:
(a) culturing a host cell according to claim 29 under conditions appropriate to cause expression of said protein; and
(b) obtaining said expressed protein form the host cell.

31. A kit for use in a method of detecting antibodies to hepatitis E virus in a biological sample, said kit comprising a swine hepatitis E virus open-reading frame 2 protein consisting of amino acids 112-660.

32. The kit of claim 32, where the protein consists of amino acids 112-660 of SEQ ID NO:1.


SEQUENCE LISTING (2 sequences)

SEQ ID NO: 1 - protein, 660 aa, swine hepatitis E virus
MRPRAVLLLLFVLLPMLPAPPAGQPSGRRCGRRNGGAGGGFWGDRVDSQPFALPYIHPTNPFAADVVSQPGAGVRPRQPPRPLGSAWRDQSQRPSTAPRRRSAPAGAAPLTAVSPAPDTAPVPDVDSRGAILRRQYNLSTSPLTSSVAAGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTVDYPARAHTFDDFCPECRTLGLQGCAFQSTIAELQRLKMKVGKTRES

SEQ ID NO: 2 - DNA, 1980 bp, swine hepatiris E virus
atgcgccctagggctgttctgttgttgctcttcgtgcttctgcctatgctgcccgcgccaccggccggccagccgtctggccgccgttgtgggcggcgcaacggcggtgccggcggtggtttctggggtgacagggttgattctcagcccttcgccctcccctatattcatccaaccaaccccttcgctgccgatgtcgtttcacaacccggggctggagttcgccctcgacagccgccccgcccccttggctccgcttggcgtgaccagtcccagcgcccctccactgccccccgtcgtcgatctgccccagctggggctgcgccgctgactgctgtatcaccggcccccgacacagctcctgtacctgatgttgactcacgtggtgctatcctgcgccggcagtacaatctgtctacgtccccgctcacgtcatctgtcgctgctggtaccaacctggttctctatgccgccccgctgaatcctctcttgcccctccaggatggcaccaacactcatattatggctactgaggcgtccaattatgctcagtatcgggttgttcgagctacgatccgttatcgcccgctggtgccaaatgctgttggtggctatgctatctctatttctttctggcctcaaactacaaccacccctacttcagttgacatgaactctattacctccactgatgtcaggattttggttcagcccggtattgcctccgagttagtcatccctagtgagcgccttcattaccgcaatcaaggctggcgctctgtagagaccacgggcgtggccgaggaggaagctacctccggtctggtaatgctttgcattcacggttctcctgttaactcctatactaacacaccttacactggtgcattggggctccttgattttgcattagagcttgaattcagaaatttgacacccgggaacactaacacccgtgtttcccggtacaccagcacagcccgccatcggctgcgccgcggtgctgatgggaccgcagagcttaccaccacagcagccacacgtttcatgaaggacttgcatttcaccggcacgaacggcgttggtgaggtgggtcgcggtatagctctaacactgtttaaccttgctgatacgcttcttggtggtttaccgacagaattgatttcgtcggccgggggccaactgttttactcccgccctgtcgtctcggccaatggcgagccgacggttaagttatatacatctgttgagaatgcgcagcaggacaagggcattaccatcccacacgatatagatctgggtgattcccgtgtggttattcaggattatgataaccagcacgagcaagaccgacctactccgtcaccagccccctctcgccctttctcagttcttcgcgccaatgatgttctgtggctctccctcaccgccgctgagtacgatcagactacatatgggtcgtccaccaaccctatgtatgtctccgatacggtcacgctagttaatgtggccactggtgctcaggctgttgcccgctctcttgattggtctaaagtcactctggatggccgccccctcactaccattcagcagtattcaaagacattctatgttctcccgctccgcgggaagctgtccttttgggaggctggtaccactaaggccggctacccgtataattataataccactgctagtgatcaaattttgattgagaacgcggctggccaccgtgttgctatctctacctataccactagcttgggtgccggccctacctcgatttccgccgttggtgtgctagccccacactcggctctcgccgtccttgaggatactgttgattaccctgctcgtgctcatacttttgatgatttctgcccggagtgccgcacccttggtttgcagggttgtgcattccagtctactattgctgagcttcagcgtcttaaaatgaaggtaggtaaaacccgggagtct